(19) United States
(12) Patent Application Publication
(10) Pub. No.: US 2013/0122048 A1
(43) Pub. Date: May 16, 2013

(54) Tumor Cell-Based Cancer Immunotherapeutic Compositions and Methods

(71) Applicants: Tzyy-Choou Wu, Stevenson, MD (US); Chien-Fu Hung, Timonium, MD (US)

(72) Inventors: Tzyy-Choou Wu, Stevenson, MD (US); Chien-Fu Hung, Timonium, MD (US)

(21) Appl. No.: 13/475,436

(22) Filed: May 18, 2012

Related U.S. Application Data
(63) Continuation of application No. 12/386,490, filed on Apr. 17, 2009, now abandoned.
(60) Provisional application No. 61/124,503, filed on Apr. 17, 2008.

(51) Int. Cl. A61K 39/00

(52) U.S. Cl. CPC A61K 39/0011
USPC 424/277.1

(57) ABSTRACT

The present invention is based, in part, on the discovery that immunotherapy using cell-based tumor cells genetically modified to express heat shock proteins is particularly effective in preventing, prognosing and/or treating cancer (e.g., ovarian cancer). Accordingly, the invention relates to compositions, kits, and methods for preventing, prognosing and/or treating cancer (e.g., ovarian cancer).

### RELATED APPLICATIONS

This application claims the benefit of U.S. Provisional Application Ser. No. 61/124,503, filed on Apr. 17, 2008; the entire contents of which is incorporated herein by reference.

### GOVERNMENT FUNDING

Work described herein was supported, at least in part, by the National Institutes of Health (NIH) under grant 1U19 CA 113341-01. The government may therefore have certain rights to this invention.

### BACKGROUND OF THE INVENTION

Immunotherapy is a plausible approach for the prevention, prognosing and/or treatment of cancer because of its specificity, sensitivity, potency, and long-term memory. Induction of a T lymphocyte response is a critical initial step in a host's immune response. The ideal cancer therapy should have the potency to direct these immunological mechanisms to eradicate systemic tumors at multiple sites in the body, as well as, the specificity to discriminate between malignant and normal cells. In both of these respects, the immune system is an attractive candidate.

B and T cells can generate tumor-specific responses because they have a vast array of clonally distributed antigen receptors, which can recognize antigens expressed only by tumors. Activation of T cells results in T cell proliferation, cytokine production by T cells and generation of T cell-mediated effector functions. T cell activation requires an antigen-specific signal, often called a primary activation signal, which results from stimulation of a clonally-distributed T cell receptor (also referred to herein as TcR) present on the surface of the T cell. This antigen-specific signal is usually in the form of an antigenic peptide bound either to a major histocompatibility complex (also referred to herein as MHC) class I protein or an MHC class II protein present on the surface of an antigen presenting cell (also referred to herein as APC). CD4⁺ T cells recognize peptides associated with class II molecules. Class II molecules are found on a limited number of cell types, primarily B cells, monocytes/macrophages and dendritic cells, and, in most cases, present peptides derived from proteins taken up from the extracellular environment. In contrast, CD8⁺ T cells recognize peptides associated with class I molecules. Class I molecules are found on almost all cell types and, in most cases, present peptides derived from endogenously synthesized proteins.

Despite an understanding of basic immunological concepts, therapies that augment the host immune response have not generally been applied to patients with cancer. For example, there is an emerging need for innovative therapies for the control of advanced ovarian cancer. Ovarian cancer is responsible for the highest mortality rate among patients with gynecologic malignancies. Metastatic ovarian cancer is extremely difficult to cure and accounts for ˜20% of total cancer mortalities among women. Current efforts to reduce this mortality rate, including improvements in early detection and treatment, have been relatively unsuccessful. Existing standard therapies for advanced disease, such as primary cytoreductive surgery followed by chemotherapy, rarely result in long-term benefits for patients with locally advanced and metastatic disease (Pfisterer and Ledermann (2006) Semin. Oncol. 33, S12-16; Bhoola and Hoskins (2006) Obstet. Gynecol. 107, 1399-1410; Ozols (2006) Semin. Oncol. 33, S3-11).

While the identification of an alternative approach to control cancer (e.g., ovarian cancer) represents an urgent concern, effective immunotherapies have been limited by a number of factors, including the ability to target specific cancer antigens, the lack of cancer models, and the difficulty in assessing tumor loads of subjects. Accordingly, there exists a need in the art to develop cell-based tumor vaccines that are effective for preventing, prognosing and/or treating cancer (e.g., ovarian cancer) in subjects.

### SUMMARY OF THE INVENTION

The present invention is based, in part, on the discovery that immunotherapy using cell-based tumor cells genetically modified to express heat shock proteins is particularly effective in preventing, prognosing and/or treating cancer (e.g., ovarian cancer). Accordingly, the invention relates to compositions, kits, and methods for preventing, prognosing and/or treating cancer (e.g., ovarian cancer).

In one aspect, the present invention provides a tumor cell-based vaccine comprising tumor cells that are genetically modified to constitutively express at least one heat shock protein.

In another aspect, the present invention pertains to an in vitro or ex vivo method of generating a tumor cell-based vaccine that treats primary or metastatic cancer in a subject, the method comprising, providing tumor cells from the subject and genetically modifying the tumor cells to constitutively express at least one heat shock protein.

In still another aspect, the present invention provides a kit comprising a tumor cell-based vaccine of the invention and instructions for use.

In yet another aspect, the present invention provides a method of treating primary or metastatic cancer in a subject, the method comprising administering a tumor cell-based vaccine of the present invention in a therapeutically effective amount.

In another aspect, the present invention further provides a method for monitoring the progression of cancer in a subject, the method comprising the steps of a) administering to the subject at a first point in time a tumor cell-based vaccine of the present invention; b) detecting in a subject sample at a subsequent point in time the number of tumor cells of the vaccine in a); and c) comparing the number of tumor cells of the vaccine in a) detected in steps a) and b) to monitor the progression of the immune disorder. In one embodiment, a significantly higher number of tumor cells of the vaccine in a) detected in step a) compared to step b) is an indication that the cancer has progressed. In another embodiment a significantly lower or unchanged number of tumor cells of the vaccine in a) detected in step a) compared to step b) is an indication that the cancer has regressed. In still another embodiment, the subject has undergone treatment to ameliorate the cancer in between the first point in time and the subsequent point in time. In yet another embodiment, the cancer is ovarian cancer.

Pertaining to any of the compositions, methods, or kits of the present invention, the following embodiments are contemplated. In one embodiment, the tumor cells are genetically modified by introducing a vector comprising nucleotide sequences encoding for at least one heat shock protein (e.g., hsp70, gp96, and gp170). In another embodiment, the at least one heat shock protein is a fusion protein (e.g., comprising at least one of a secretion signal, cleavage, or reporter sequence). In still another embodiment, the vector is a recombinant retrovirus. In yet another embodiment, the tumor cells are ovarian cancer tumor cells (e.g., mouse or human tumor ovarian tumor cells such as MOSEC or ovcar3 cells). In other embodiments, the ovarian cancer tumor cells are autologous, xenogeneic, allogeneic or syngeneic to the subject. In another embodiment, the tumor cells are non-replicative (e.g., due to irradiation). In still another embodiment, the tumor cell-based vaccine can inhibit tumor growth or stimulates tumor-specific CD8+ T cells in a subject.

### BRIEF DESCRIPTION OF THE DRAWINGS

FIGS. 1A-1D show the results of in vivo tumor growth experiments in mice challenged with MOSEC/luc cells expressing either GFP or Hsp70-GFP following characterization of MOSEC/luc cell line transduced with sHsp70-T2A-GFP or T2A-GFP. FIG. 1A shows flow cytometry analysis showing GFP expression level in MOSEC/luc cells transduced with retrovirus containing either sHsp70-T2A-GFP (Hsp70-GFP) or T2A-GFP (GFP). The untransfected cells were used as a control. FIG. 1B shows Western blot analysis showing the expression of secreted mouse Hsp70 protein. The supernatant from the culture medium of luciferase-expressing MOSEC cells (MOSEC/luc) transfected with Hsp70-GFP (lane 1) or GFP (lane 2) was used for Western blot analysis using antibody specific to Hsp70. In lane 1, the higher molecular weight band (−100 kDa) represents the uncleaved fusion protein of sHsp70 and EGFP. FIG. 1C shows luminescence images of representative C57BL/6 mice (five per group) challenged with MOSEC/luc cells expressing Hsp70-GFP or GFP on days 0 (D0), 7 (D7), and 14 (D14) after tumor challenge. FIG. 1D shows quantification of luminescent activity in mice challenged with MOSEC/luc cells transfected with Hsp70-GFP or GFP on days 0, 7, and 14. The mean and standard deviation are indicated. C57BL/6 mice (five per group) were intraperitoneally (i.p.) challenged with 1×10⁶ per mouse of viable MOSEC/luc cells expressing either Hsp70-GFP or GFP. Bioluminescence signals were acquired for 1 min. using the IVIS Imaging System Series 200™. The P values are shown and the groups with statistical significance (P<0.05) are indicated by asterisks.

FIGS. 2A-2D show the results of in vivo tumor protection experiments in mice immunized i.p. with live or irradiated MOSEC/luc cells expressing Hsp70-GFP or GFP. C57BL/6 mice (five per group) that were previously challenged with MOSEC/luc expressing Hsp70-GFP were rechallenged i.p. after 2 wk with 1×10⁶ per mouse of MOSEC/luc cells. As a control, a group of naive mice was challenged with 1×10⁶ per mouse of live MOSEC/luc cells.

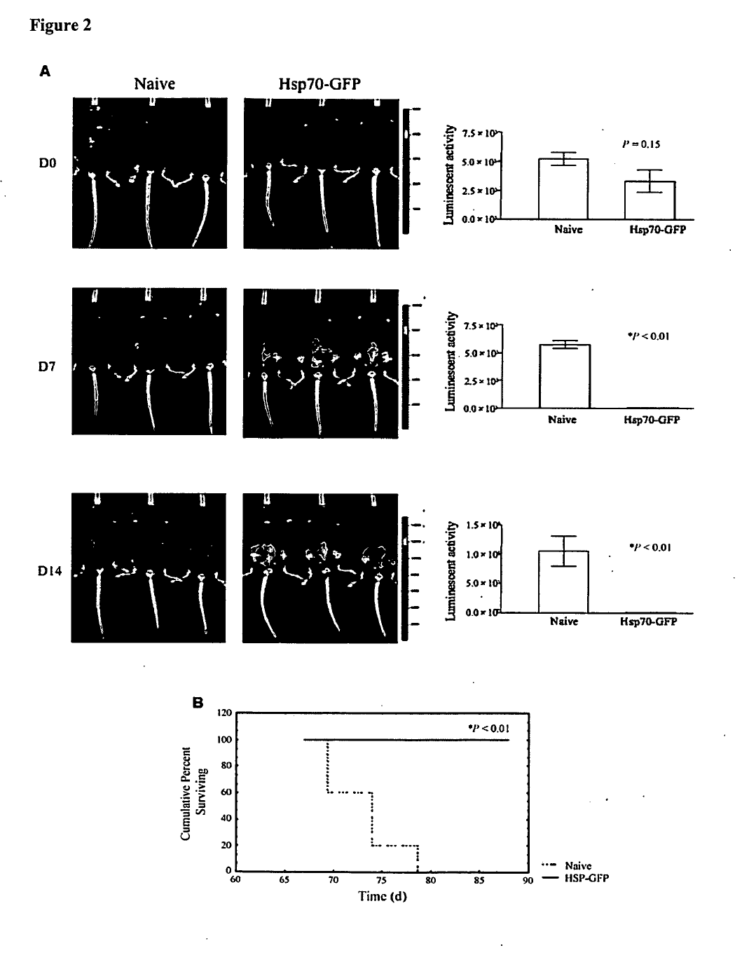

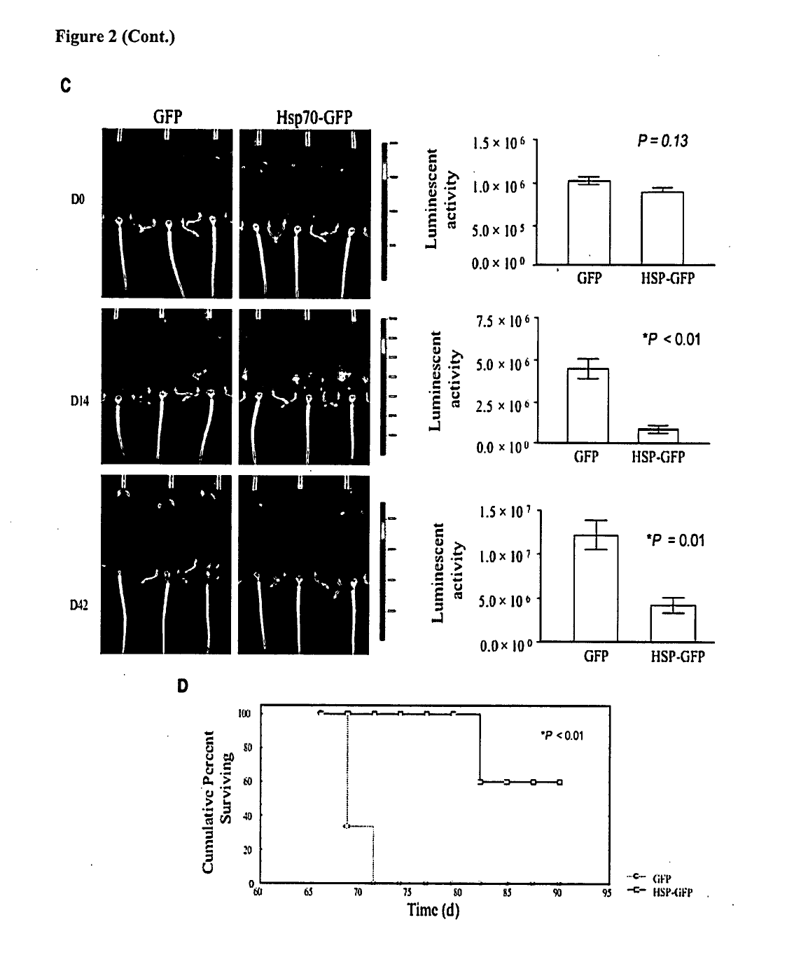

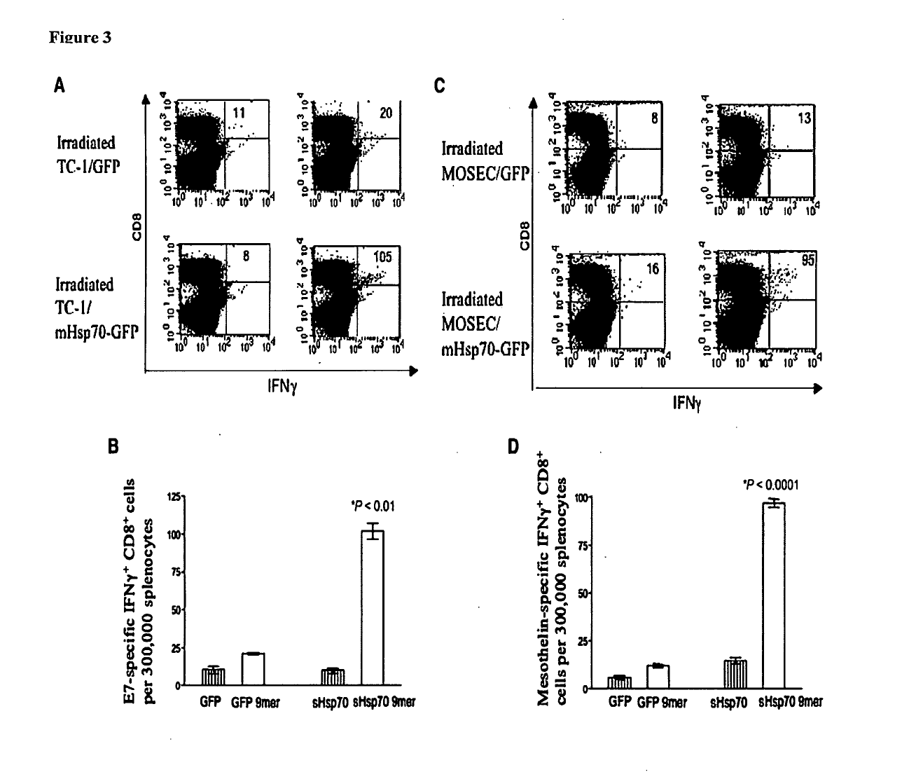

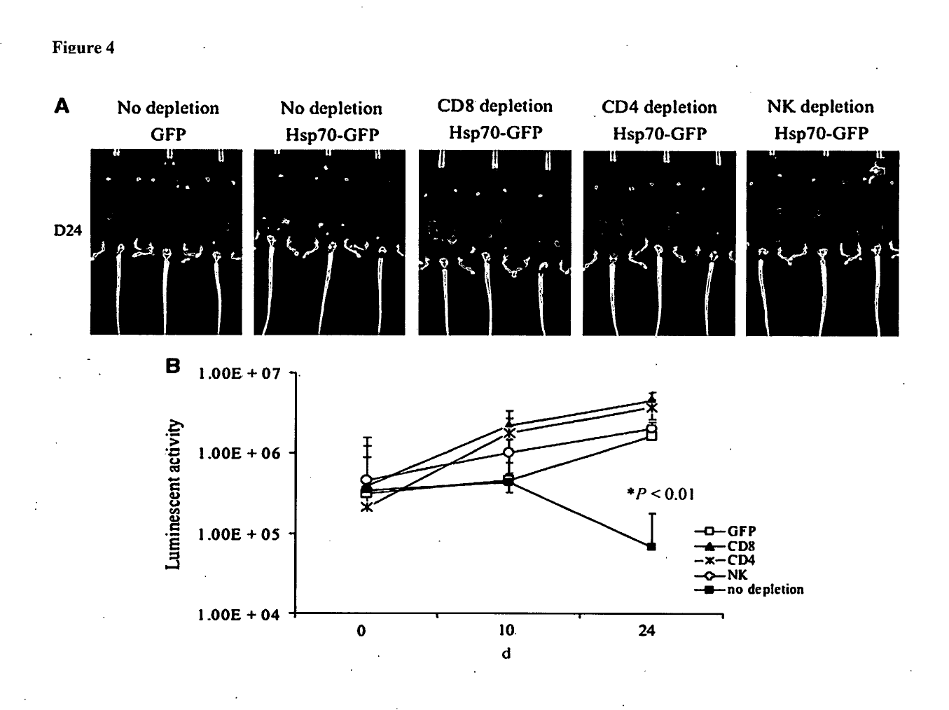

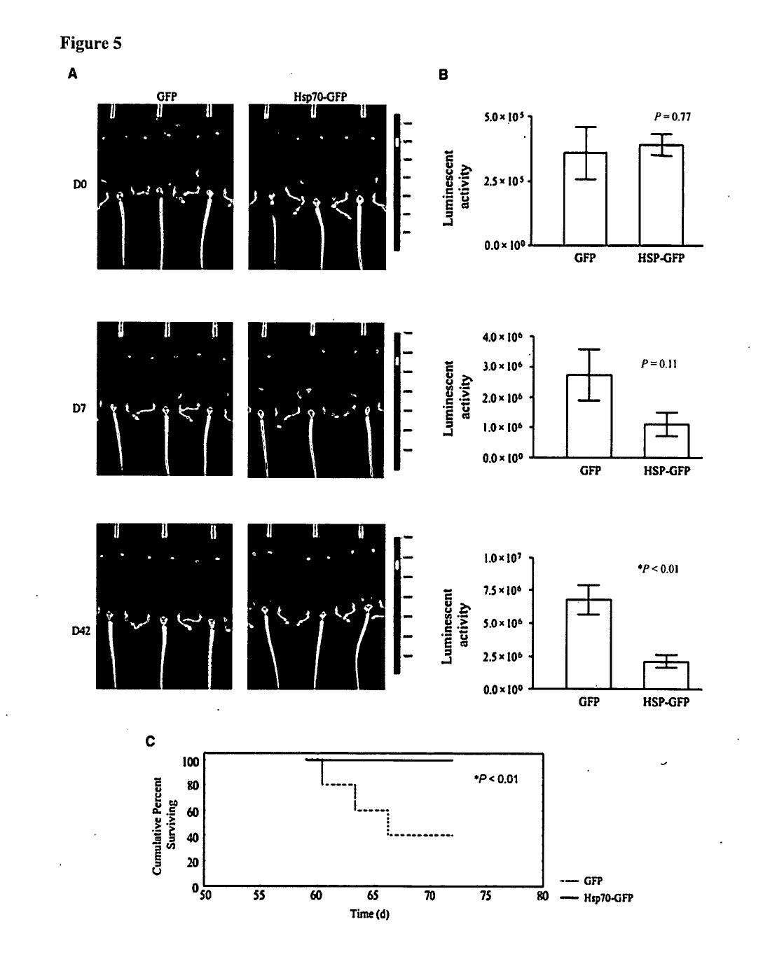

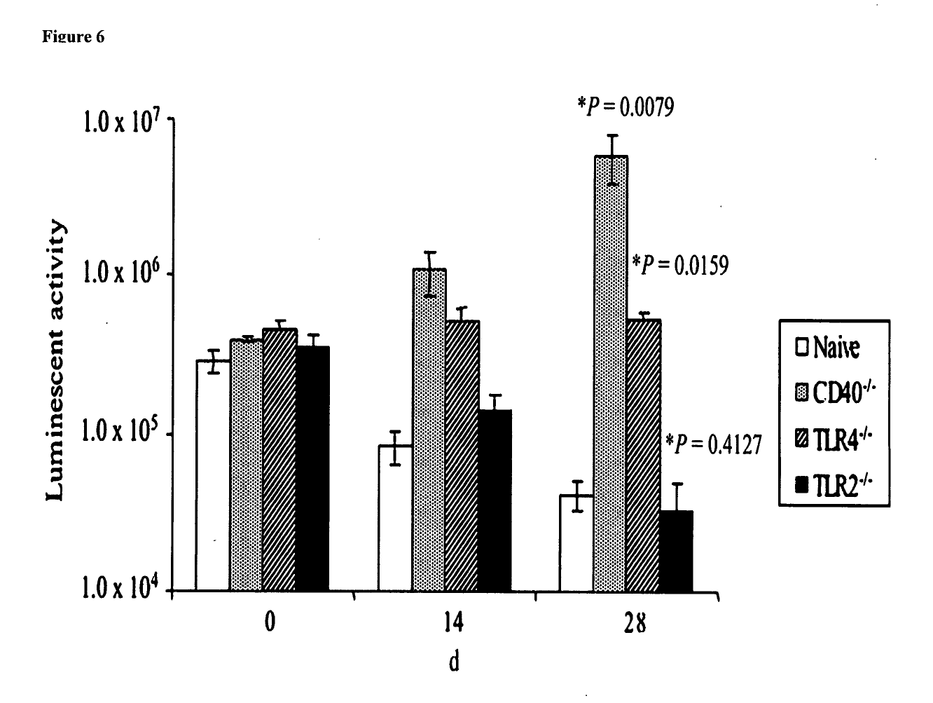

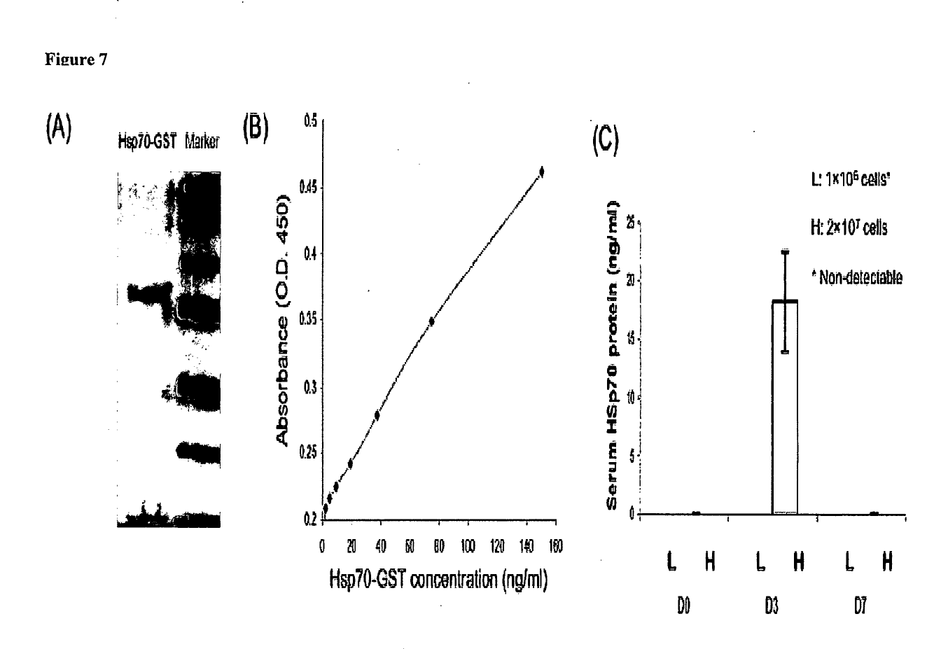

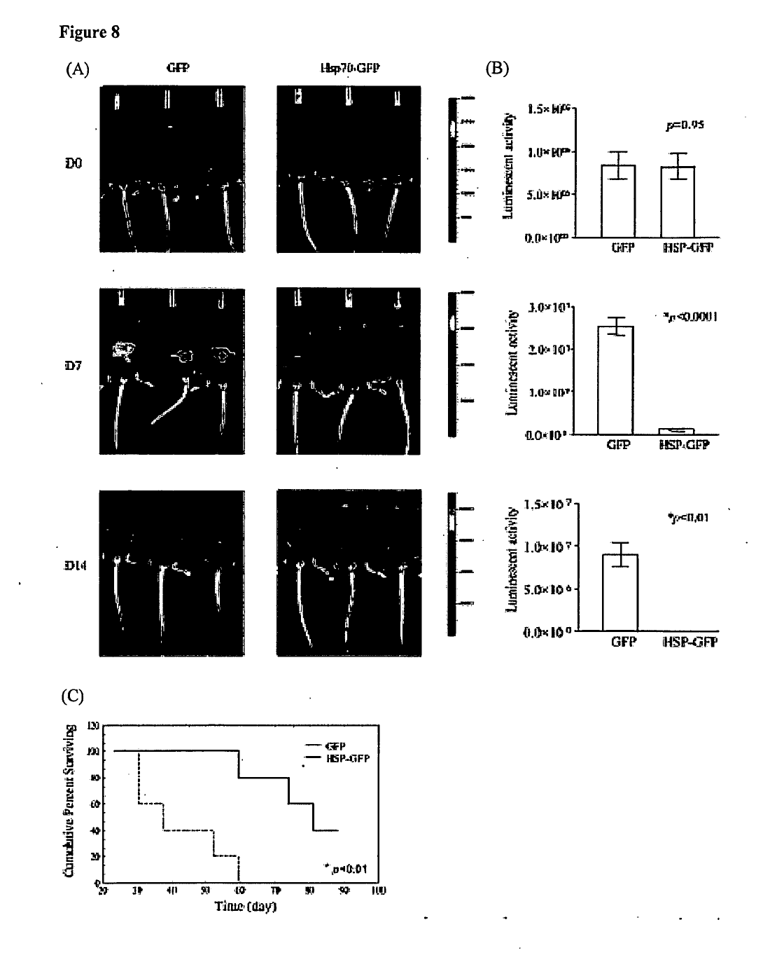

Mice were imaged using the IVIS Imaging System Series 200™ and bioluminescence signals were acquired for 1 min. FIG. 2A shows representative luminescence images of naive mice challenged with MOSEC/luc cells and mice rechallenged with MOSEC/luc cells on days 0, 7, and 14, as well as quantification of luminescent activity in naive mice or mice rechallenged with MOSEC/luc cells on days 0, 7, and 14. The mean and standard deviation are indicated. FIG. 2B shows the results of Kaplan-Meier survival analysis indicating long-term survival in mice rechallenged with MOSEC/luc cells compared with naive mice control. C57BL/6 mice (five per group) were also immunized with 1×10⁶ per mouse of irradiated MOSEC/luc cells expressing Hsp70-GFP or GFP. Two weeks after immunization, the vaccinated mice were challenged with 1×10⁶ per mouse of MOSEC/luc cells. Mice were imaged using the IVIS Imaging System Series 200™ and bioluminescence signals were acquired for 1 min. FIG. 2C shows luminescence images of representative mice immunized with irradiated MOSEC/luc cells expressing Hsp70-GFP or GFP on days 0, 14, and 42, as well as quantification of luminescent activity in mice immunized with irradiated MOSEC/luc cells expressing Hsp70-GFP or GFP on days 0, 14, and 42. The mean and standard deviation are indicated. FIG. 2D shows the results of Kaplan-Meier survival analysis indicating long-term survival in mice immunized with MOSEC/luc cells expressing Hsp70-GFP compared with MOSEC/luc cells expressing GFP. The P values are shown and the groups with statistical significance (P<0.05) are indicated by asterisks.

FIG. 3A-3D show the results of flow cytometry analysis of IFN-γ-secreting antigen-specific CD8⁺ cell precursors in mice vaccinated with TC-1 or MOSEC cell-based vaccines. In FIGS. 3A and 3B, C57BL/6 mice (five per group) were vaccinated i.p. with 1×10⁶ per mouse of irradiated TC-1/luc cells expressing either Hsp70-GFP or GFP twice with a 1-wk interval. In FIGS. 3C and 3D, C57BL/6 mice were vaccinated i.p. with 1×10⁶ per mouse of irradiated MOSEC/luc cells expressing Hsp70-GFP or GFP twice with a 1-wk interval. Determination of the CD8 cells was done by culturing the splenocytes from vaccinated mice with E7 peptide (FIGS. 3A and 3B) or mesothelin peptide (FIGS. 3C and 3D) and analyzed for CD8 and intracellular IFN-γ staining by flow cytometry. FIG. 3A shows representative flow cytometry data indicating the number of E7-specific IFN-γ CD8⁺ T cells in the mice vaccinated with irradiated TC-1/luc cells expressing Hsp70-GFP or GFP. FIG. 3B shows the number of IFN-γ⁺ CD8⁺ T cells from each group with (white columns) or without (shaded columns) stimulation by the E7 peptide. The mean and standard deviations are indicated. FIG. 3C shows representative flow cytometry data showing the number of mesothelin-specific IFN-γ⁺ CD8⁺ T cells in mice vaccinated with irradiated MOSEC/luc cells expressing either Hsp70-GFP or GFP. FIG. 3D shows the number of mesothelin-specific IFN-γ⁺ CD8⁺ T cells from each group with (white columns) or without (shaded columns) stimulation by the mesothelin peptide. The mean and standard deviations are indicated. The P values are also shown and the groups with statistical significance (P<0.05) are indicated by asterisks.

FIGS. 4A-4B show the results of in vivo antibody depletion experiments. C57BL/6 mice (five per group) were i.p. immunized with 1×10⁶ per mouse of irradiated MOSEC/luc cells expressing Hsp70-GFP or GFP. Two weeks after vaccination, the immunized mice were challenged with 1×10⁶ per mouse of MOSEC/luc cells. One week after the vaccination, the Hsp70-GFP-vaccinated mice were depleted of CD8, CD4, or NK cells using 100 μg each of purified rat mAbs 2.43, 100 GK1.5, and PK136, respectively. The mice were injected with antibodies every other day for three times for the first week and then once every week using a protocol similar to one described in Chen et al. (2000) Cancer Res. 60, 1035-1042. Mice were imaged using the IVIS Imaging System Series 200™ and bioluminescence signals were acquired for 1 min. FIG. 4A shows luminescence images in representative mice challenged with MOSEC/luc cells expressing Hsp70-GFP with CD4 depletion, CD8 depletion, or NK depletion and MOSEC/luc cells expressing GFP. FIG. 4B shows quantification of luminescent activity in the tumors of mice challenged with MOSEC/luc cells expressing Hsp70-GFP with CD4 depletion, CD8 depletion, or NK depletion and MOSEC/luc cells expressing GFP. The P values are shown and the groups with statistical significance (P<0.05) are indicated by asterisks.

FIGS. 5A-5C show the results of in vivo tumor treatment. C57BL/6 mice (five per group) were i.p. challenged with 1×10⁶ per mouse of MOSEC/luc cells. Five days later, the mice were treated with 1×10⁶ per mouse of irradiated MOSEC/luc cells expressing either Hsp70-GFP or GFP. Mice were imaged using the IVIS Imaging System Series 200™ and bioluminescence signals were acquired for 1 min. FIG. 5A shows luminescence images in representative mice treated with MOSEC/luc cells expressing Hsp70-GFP or GFP on days 0, 7, and 42. FIG. 5B shows quantification of luminescent activity in mice treated with MOSEC/luc cells expressing Hsp70-GFP or GFP on days 0, 7, and 42. FIG. 5C shows the results of Kaplan-Meier survival analysis showing long-term survival in mice treated with irradiated MOSEC/luc cells expressing Hsp70-GFP compared with the mice treated with the irradiated MOSEC/luc cells expressing GFP. The P values are shown and the groups with statistical significance (P<0.05) are indicated by asterisks.

FIG. 6 shows the results of in vivo tumor growth experiments in CD40, TLR2, and TLR4 knockout mice challenged with MOSEC/luc cells expressing Hsp70-GFP. Quantification of tumor load (luminescent activity) was performed in CD40, TLR2, or TLR4 knockout C57BL/6 mice (five per group) challenged with viable MOSEC/luc cells expressing Hsp70-GFP on days 0, 14, and 28. Naive C57BL/6 mice challenged with MOSEC/luc cells expressing Hsp70-GFP were used as control. C57BL/6 mice (five per group) were i.p. challenged with 1×10⁶ per mouse of the viable Hsp70-secreting tumor cells. Bioluminescence signals were acquired for 1 min. The mean and standard deviations are indicated. The P values are shown and the groups with statistical significance (P<0.05) are indicated by asterisks.

FIGS. 7A-7C show the results of characterizing the in vitro and serum concentrations of Hsp70. FIG. 7A shows a purified GST-Hsp70 protein band (lane 1) as assayed by Coomassie Blue staining of an SDS gel. FIG. 7B shows the results of an ELISA assay to generate a standard curve of GST-Hsp70 protein concentration. A linear relationship of the absorbance read at 450 nm to purified Hsp70 protein concentrations was observed. FIG. 7C shows a bar graph indicating the serum concentrations of Hsp70 detected by ELISA. Mice were immunized with 1×10⁶ or 2×10⁷ MOSEC-luc/sHsp70-GFP or MOSEC-luc/GFP cells intraperitoneally. Serum samples were taken on D0, D3, and D7.

FIGS. 8A-8C show the results of in vivo tumor growth experiments in mice challenged with TC-1 cells expressing either Hsp70-GFP or GFP. C57BL/6 mice (five per group) were intraperitoneally challenged with 1×10⁶ per mouse of viable luciferase expressing TC-1 cells (TC-1/luc) expressing either Hsp70-GFP or GFP. Tumor challenged mice were imaged using the IVIS Imaging System Series 200™. FIG. 8A shows luminescence images in representative mice treated with TC-1/luc cells expressing Hsp70-GFP or GFP on D0 (P<0.01), D7 (P<0.0001) and D14 (P<0.01) after tumor challenge. FIG. 8B shows bar graphs depicting the quantification of luminescent activity in mice challenged with TC-1/luc cells expressing Hsp70-GFP or GFP on D0, D7, and D14. The data is shown as the mean±standard deviation. FIG. 8C shows the results of Kaplan-Meier survival analysis showing long-term survival in mice challenged with TC-1/luc cells expressing Hsp70-GFP compared to the TC-1/luc cells expressing GFP (P<0.01).

### DETAILED DESCRIPTION OF THE INVENTION

The invention is based, in part, on the discovery that immunotherapy using cell-based tumor cells genetically modified to express heat shock proteins is particularly effective in preventing, prognosing, and treating cancer (e.g., ovarian cancer). Accordingly, the invention relates to compositions, kits, and methods for preventing, prognosing and/or treating cancer (e.g., ovarian cancer).

### I. Definitions

The articles “a” and “an” are used herein to refer to one or to more than one (i.e. to at least one) of the grammatical object of the article. By way of example, “an element” means one element or more than one element.

The term “allogeneic” as used herein is defined as a material derived from the same animal species but genetically different in one or more genetic loci as the animal that becomes the recipient. This usually applies to tumor cells transplanted from one animal to another non-identical animal of the same species.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response.

The term “antigen presenting cell” (APC) as used herein, is defined as a cell that is capable of activating T cells or other immune cells, and includes, but is not limited to, monocytes/macrophages, B cells and dendritic cells (DCs).

The term “autologous” as used herein is defined as material derived from the same individual to whom it is later to be re-introduced therein.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, and vomit).

The term “cancer” as used herein is defined as a disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, ocular cancer, pancreatic cancer, colorectal cancer, renal cancer, liver cancer, brain cancer, lymphoma, leukemia, lung cancer and the like.

In certain embodiments of the present invention, the cancer is ovarian cancer. Ovarian cancer can be staged according to the AJCC/TNM System that describes the extent of the primary tumor (T), the absence or presence of metastasis to nearby lymph nodes (N), and the absence or presence of distant metastasis (M). This closely resembles the system that is actually used by most gynecologic oncologists, called the FIGO system. “Advanced epithelial ovarian cancer”, as used herein, includes patients with stage III or stage IV ovarian cancer. More particularly, and in one embodiment, the term includes patients with stage IIIc or stage IV ovarian cancer, determined according to a recognized staging technique such as the AJCC/TMN or FIGO system. In patients diagnosed with stage III ovarian cancer the cancer involves one or both ovaries, and one or both of the following are present: (1) cancer has spread beyond the pelvis to the lining of the abdomen; (2) cancer has spread to lymph nodes. In stage IIIC patients, the cancer is in one or both ovaries, and one or both of the following are present: (1) cancer has spread to lymph nodes, and (2) deposits of cancer larger than 2 cm across are present in the abdomen. Patients diagnosed with stage IV have cancer in one or both ovaries. Distant metastasis (spread of the cancer to the inside of the liver, the lungs, or other organs located outside of the peritoneal cavity) has occurred. A finding of ovarian cancer cells in pleural fluid (from the cavity that surrounds the lungs) is also evidence of stage IV disease.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “combination therapy” as used herein is defined as combining the methods and immunovaccines of the present invention with other methods of cancer treatment. Examples of such methods include radiation, surgery and chemotherapy.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, “constitutive expression” refers to expression using a constitutive or regulated promoter. “Conditional” and “regulated expression” refers to expression controlled by a regulated promoter. A “constitutive promoter” refers to a promoter that is able to express the open reading frame (ORF) that it controls in the desired host cell at all or nearly all times for at least 1% of the level reached in cells where transcription is most active. In some embodiments, the use of the term, “constitutive” refers to the expression of the ORF regardless of the regulatory constraints normally fixing the ORF's expression (i.e., expression of ORFs encoding heat shock proteins in the absence of stressful conditions).

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes T cell mediated and/or B cell mediated immune responses. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages.

As used herein, the term “inhibit” includes the decrease, limitation, or blockage, of, for example a particular action, function, or interaction.

As used herein, the term “in vivo diagnostics” refers to in vivo imaging methods, which permit the detection of a labeled molecule that is specifically produced by cells (e.g., tumor cells) in the subject's body. Such methods include detection of bioluminescence or fluorescence, magnetic resonance imaging (MRI), positron-emission tomography (PET) and single photon emission tomography (SPECT).

As used herein, the term “interaction,” when referring to an interaction between two molecules, refers to the physical contact (e.g., binding) of the molecules with one another. Generally, such an interaction results in an activity (which produces a biological effect) of one or both of said molecules.

A “kit” is any manufacture (e.g. a package or container) comprising at least one reagent, e.g., a probe, for specifically detecting the expression of a marker of the invention. The kit may be promoted, distributed, or sold as a unit for performing the methods of the present invention.

A “marker” is a gene whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker of the invention. Such marker nucleic acids include DNA (e.g., cDNA) comprising the entire or a partial sequence of any of the nucleic acid sequences set forth in the Sequence Listing or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” is a protein encoded by or corresponding to a marker of the invention. A marker protein comprises the entire or a partial sequence of any of the sequences set forth in the Sequence Listing. The terms “protein” and “polypeptide” are used interchangeably.

The “normal” level of expression of a marker is the level of expression of the marker in cells of a subject, e.g., a human patient, not afflicted with cancer, e.g., ovarian cancer. An “over-expression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples. A “significantly lower level of expression” of a marker refers to an expression level in a test sample that is at least twice, and more preferably three, four, five or ten times lower than the expression level of the marker in a control sample (e.g., sample from a healthy subject not having the marker associated disease) and preferably, the average expression level of the marker in several control samples.

As used herein, the term “preventing” or “prevention” refers to delaying or forestalling the onset, development or progression of a condition or disease for a period of time, including weeks, months, or years.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

As used herein, “significantly higher” refers to a difference of a measured parameter (e.g., cell amount) in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than that in a control sample (e.g., sample from a subject at the time point of administration with a composition of the invention) and preferably, the average over several control samples. “Significantly lower” refers to a difference of a measured parameter (e.g., cell amount) in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a subject at the time point of administration with a composition of the invention) and preferably, the average over several control samples.

As used herein, the term “subject” or “patient” refers to a human or non-human animal selected for treatment or therapy.

As used herein, the term “subject suspected of having” refers to a subject exhibiting one or more clinical indicators of a disease or condition. In certain embodiments, the disease or condition is cancer. In certain embodiments, the cancer is ovarian cancer.

As used herein, the term “subject in need thereof” or refers to a subject identified as in need of a therapy or treatment.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

The term “syngeneic” as used herein is defined as a material derived from the same animal species and has the same genetic composition for most genotypic and phenotypic markers as the recipient.

The term “T-cell” as used herein is defined as a thymus-derived cell that participates in a variety of cell-mediated immune reactions. Most of the T cells in the body belong to one of two subsets. These are distinguished by the presence on their surface of one or the other of two glycoproteins designated: CD4 and CD8. Which of these molecules is present determines what types of cells the T cell can bind to. CD8⁺ T cells bind epitopes that are part of class I histocompatibility molecules. Almost all the cells of the body express class I molecules. CD4⁺ T cells bind epitopes that are part of class II histocompatibility molecules. The best understood CD8⁺ T cells are cytotoxic T lymphocytes (CTLs). They secrete molecules that destroy the cell to which they have bound. CD4⁺ T cells bind an epitope consisting of an antigen fragment lying in the groove of a class II histocompatibility molecule. CD4⁺ T cells are essential for both the cell-mediated and antibody-mediated branches of the immune system. Activated CD4⁺ T cells are either Type 1 (Th1) or Type 2 (Th2), or Type 17 (Th17) based on their cytokine secretion profile. Type 1 cells secrete IL-2, IFN-γ, TNF-α, GM-CSF, and; Th2 cells secrete, IL-4, IL-5, IL-10, and IL-13. Type 1 CD4⁺ T-cells, which secrete IFN-γ, are a critical component for the activation of CD8⁺ T cells, either through the “helper” T cells that provide cytokine support for CD8⁺ T cells or by the induction of CD40 on dendritic cells which in turn activate CD8⁺ T cells. CD4⁺ T cells are essential for generating CD8⁺ T memory cells, for preventing CD8⁺ T cells from being tolerized and for recruiting cells of the innate immue system. Type 1 cells provide help to cytotoxic CD8⁺ T cells, Type 2 cells facilitate antibody production by B lymphocytes, while Type 3 cells produce IL-17. It is believed that immune responses skewed toward CD4⁺ Type 1 cells and away from Type 2 responses are optimal for antitumor immunity because CD8-mediated killing is highly efficient for destroying tumor cells. Further, Type 1 cytokine IFN-γ plays an important role in regulating in vivo tumor growth by both the innate and adaptive immune systems. IFN-γ is a pleiotropic cytokine that has many effects ranging from stimulation of T cell-mediated and NK responses to enhancing MHC class I and class II expression on target cells.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

The term “transfected,” “transformed,” or “transduced” as used herein refers to a process by which exogenous nucleic acid is transferred or introduced into the host cell. A “transfected,” “transformed” or “transduced” cell is one which has been transfected, transformed or transduced with exogenous nucleic acid. The cell includes the primary subject cell and its progeny.

As used herein, the term “treatment” or “treat” means the application of one or more procedures used for the amelioration of a disease. In certain embodiments the specific procedure is the administration of one or more pharmaceutical agents.

The term “vaccine” or “immunovaccine” as used herein is defined as a composition that can elicit a detectable immune response when administered to an animal. In some embodiments, an immunovaccine stimulates and activates T cells when administered to the animal, such that it generates a detectable T cell immune response to an antigen, a tumor cell, and the like, when compared to a T cell, the immune response, if any, in an otherwise identical animal to which the immunovaccine is not administered. For examples, in some embodiments, an immunovaccine comprises an engineered tumor cell (e.g., tumor cell genetically engineered to express a heat shock protein).

As used herein, the term “vector” refers to a nucleic acid capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “xenogeneic” as used herein is defined as a material derived from a different animal species than the animal species that becomes the subject of the vaccine.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for a fusion protein or a polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

### II. Heat Shock Proteins

The present invention relates, in part, to compositions and methods for the prevention, prognositcation, and treatment of cancer (e.g., ovarian cancer) using tumor cells engineered to express at least one heat shock protein (e.g., immunovaccines). These compositions and methods depend upon the use of tumor cells expressing antigens to a given cancer as well as the effect described herein of heat shock protein (HSP) expression which amplify immune responses to these tumor cell antigens. The compositions and methods are useful for improving the prevention, prognostication, and treatment outcome in a subject administered with the HSP preparation and the immunoreactive antigen source (e.g., tumor cell). The invention also provides compositions and methods useful for producing or enhancing an immune response elicited by an immunoreactive reagent, comprising the administration of an HSP preparation.

Heat shock proteins can traditionally be characterized as those proteins whose expression is increased in mammalian cells which were exposed to sudden elevations of temperature, while the expression of most cellular proteins is significantly reduced. As used herein, the term “heat shock protein” will be used to encompass both proteins that are expressly labeled as such as well as other stress proteins, including homologues of such proteins that are expressed constitutively (i.e., in the absence of stressful conditions). Examples of heat shock proteins include BiP (also referred to as grp78), hsp70, gp96 (grp94), gp 170, hsp60, hsp40 and hsp90. Naturally occurring or recombinantly derived mutants of heat shock proteins may also be used according to the invention. For example, but not by way of limitation, the present invention provides for the use of heat shock proteins mutated so as to facilitate their secretion from the cell (for example having mutation or deletion of an element which facilitates endoplasmic reticulum recapture, such as KDEL or its homologues; such mutants are described in PCT Application No. PCT/US96/13233 (WO 97/06685), which is incorporated herein by reference).

Heat shock proteins have the ability to bind other proteins in their non-native states, and in particular to bind nascent peptides emerging from ribosomes or extruded into the endoplasmic reticulum (Hendrick and Hartl, Ann. Rev. Biochem. 62:349-384 (1993); Hartl, Nature 381:571-580 (1996)). Further, heat shock proteins have been shown to play an important role in the proper folding and assembly of proteins in the cytosol, endoplasmic reticulum and mitochondria; in view of this function, they are referred to as “molecular chaperones” (Frydman et al., Nature 370:111-117 (1994); Hendrick and Hartl, Ann. Rev. Biochem. 62:349-384 (1993); Hartl, Nature 381:571-580 (1996)).

A nucleic acid encoding a heat shock protein may be operatively linked to elements necessary or desirable for expression and then used to express the desired heat shock protein as either a means to produce heat shock protein for use in a protein vaccine or, alternatively, in a nucleic acid vaccine. Elements necessary or desirable for expression include, but are not limited to, promoter/enhancer elements, transcriptional start and stop sequences, polyadenylation signals, translational start and stop sequences, ribosome binding sites, signal sequences and the like. For example, but not by way of limitation, genes for various heat shock proteins have been cloned and sequenced, including, but not limited to, gp96 (human: Genebank Accession No. X15187; Maki et al., Proc. Natl. Acad. Sci. U.S.A. 87:5658-5562 (1990); mouse: Genebank Accession No. M16370; Srivastava et al., Proc. Natl. Acad. Sci. U.S.A. 84:3807-3811 (1987)), BiP (mouse: Genebank Accession No. U16277; Haas et al., Proc. Natl. Acad. Sci. U.S.A. 85:2250-2254 (1988); human: Genebank Accession No. M19645; Ting et al., DNA 7:275-286 (1988)), hsp70 (mouse: Genebank Accession No. M35021; Hunt et al., Gene 87:199-204 (1990); human: Genebank Accession No. M24743; Hunt et al, Proc. Natl. Acad. Sci. U.S.A. 82:6455-6489 (1995)), and hsp40 (human: Genebank Accession No. D49547; Ohtsuka K., Biochem. Biophys. Res. Commun. 197:235-240 (1993)). Based upon teachings well known to the skilled artisan, an HSP of the invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

Thus, in certain embodiments, the heat shock protein of the invention may comprise HSPs including but not limited to, BiP (also referred to as grp78), hsp70, gp96 (grp94), gp 170, hsp60, hsp40 and hsp9, hsp110, or calreticulin, singly or in combination with each other. Also encompassed by the invention are HSP fusion proteins such as hsp60 fusion proteins, hsp70 fusion proteins, hsp90 fusion proteins, hsp110 fusion proteins, gp96 fusion proteins, grp170 fusion proteins or calreticulin fusion proteins. The invention further encompasses nucleic acid molecules that differ, due to degeneracy of the genetic code, from the nucleotide sequence of nucleic acid molecules encoding a protein which corresponds to a marker of the invention, and thus encode the same protein. It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including, but not limited to, HSPs of the invention, can differ from each other in a single nucleotide, or several nucleotides, and can include substitutions, deletions, and insertions of nucleotides. An allele of a gene can also be a form of a gene containing one or more mutations.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Accordingly, it should be appreciated that HSPs of the invention encompass nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

A skilled artisan will appreciate the foregoing is meant to encompass biologically active portions of HSPs of the invention, which include polypeptides comprising amino acid sequences sufficiently identical to or derived from the amino acid sequence of the protein corresponding to the full-length HSP, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention. In some embodiments, HSP polypeptides of the invention have an amino acid sequence that is substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to the full-length HSP and retain the functional activity of the protein of the corresponding naturally-occurring protein yet differ in amino acid sequence due to natural allelic variation or mutagenesis. Well known bioinformatic algorithms can be used to determine the extent of such homology (e.g., Altschul, et al. (1990) J. Mol. Biol. 215:403-410).

The invention also contemplates chimeric or fusion proteins corresponding to HSPs of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GFP, luciferase, or other marker fusion protein in which a polypeptide corresponding to an HSP of the invention is fused to the amino or carboxyl terminus of the marker sequence. Such fusion proteins can facilitate qualitative and quantitative detection of the fusion protein. For example, GFP, luciferase, or other markers can facilitate noninvasive imaging of the fusion protein in vitro, ex vivo, and/or in vivo (Hung et al. (2007) Gene Ther. 14, 20-29; Tseng et al. (2004) Nat. Biotech. 22, 70-77).

In another embodiment, the fusion protein contains a heterologous signal sequence at its amino terminus, carboxy terminus, or anywhere within the polypeptide. For example, the native signal sequence of a polypeptide corresponding to a marker of the invention can be removed and replaced with a signal sequence from another protein. For example, the immunoglobulin k-signal sequence or gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.). In still another embodiment, cleavage sequences are well known to a skilled artisan and are useful for splitting a fusion protein in desired components polypeptides.

In yet another embodiment, the fusion protein is an immunoglobulin fusion protein in which all or part of a polypeptide corresponding to an HSP of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GFP, luciferase, cleavage, or signal peptide sequence). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain. An advantage to using the secreted form of HSP in certain embodiments of the present invention is that there is a consistent availability of the HSP rather than only after apoptosis of HSP-containing cells.

In certain embodiments, the HSP of the invention comprises a single HSP, HSP complex, or FISP fusion protein. In other embodiments of the invention, the HSP preparation comprises mixtures of HSPs, HSP complexes, or HSP fusion proteins (e.g., at least two, three, four, five, six, seven, eight, nine, ten, or more HSPs).

In various embodiments, the source of the HSP is a eukaryote (e.g., a mammal, for example, a human). Accordingly, the HSP preparation used by the methods of the invention includes eukaryotic HSPs, mammalian HSPs and human HSPs. The eukaryotic source from which the HSP preparation is derived and the subject receiving the HSP preparation are preferably the same species.

### III. Expression Vectors and Tumor Cells

Another aspect of the invention pertains to vectors, preferably expression vectors, containing a nucleic acid encoding an HSP polypeptide of the invention or a portion thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art (e.g., ovarian epithelial cells under the control of the mesothelin promoter). Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

It will be appreciated by a skilled artisan that the above-referenced vectors are examples of vectors useful for the compositions and methods of the present invention and further comprise lentiviruses, oncoretroviruses, expression plasmids, adenovirus, and adeno-associated virus. Other delivery vectors that are useful comprise herpes simplex viruses, transposons, vaccinia viruses, human papilloma virus, Simian immunodeficiency viruses, HTLV, human foamy virus and variants thereof. Further vectors that are useful comprise spumaviruses, mammalian type B retroviruses, mammalian type C retroviruses, avian type C retroviruses, mammalian type D retroviruses, HTLV/BLV type retroviruses, and lentiviruses.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The host cell of the compositions and methods of the invention comprise tumor-based cells (e.g., ovarian cancer cells). In certain embodiments, the tumor-based cells are autologous, allogeneic, syngenic, or xenogeneic to the recipient. In other embodiments, the tumor-based cells can be produced, engineered, and/or maintained in vitro or ex vivo.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to an HSP polypeptide of the invention. Accordingly, the invention further provides methods for producing a polypeptide corresponding to an HSP polypeptide of the invention using the host cells of the invention. In one embodiment, the method comprises culturing the host cell of invention (into which a recombinant expression vector encoding a polypeptide of the invention has been introduced) in a suitable medium such that the HSP polypeptide of the invention is produced. In another embodiment, the method further comprises isolating the HSP polypeptide of the invention from the medium or the host cell.

### IV. Pharmaceutical Compositions

The compositions of the invention (e.g., immunovaccines) can be incorporated into pharmaceutical compositions suitable for administration to a subject. Such compositions typically comprise a tumor cell-based composition and a pharmaceutically acceptable carrier. It is also contemplated that such immunovaccines of the present invention can be used alone or in appropriate association, as well as in combination, with other pharmaceutically active compounds as are known in the art. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pursuant to the present invention, an amount of tumor-based immunotherapeutic cells effective to prevent, prognose, or treat cancer in a subject is administered. The effective amount of such cells to be administered to a mammal (e.g., a human subject) would broadly range between about 1×10³ and 1×10¹⁰ cells per subject, about 1×10⁴ and 1×10⁹, about 1×10⁵ and 1×10⁹, about 1×10⁶ and 1×10⁹, about 1×10⁷ and 1×10⁹ such as 1×10⁷, 3×10⁷, 1×10⁸, and 3×10⁸ cells per recipient. The precise amounts will depend on the severity of the disease condition being monitored, other factors, such as diet modification that are implemented, the weight, age, and sex of the subject, and other criteria, which can be readily determined according to standard good medical practice by those of skill in the art. Dosage regimens may be adjusted to provide optimum therapeutic responses. For instance, a single dose may be administered or several doses may be administered over time.

Prior to administration to the subject, the tumor-based immunotherapeutic cells can be treated to render them incapable of further proliferation in the subject, thereby preventing any possible outgrowth of the modified primary immune-privilege tumor cells. Possible treatments include irradiation or mitomycin C treatment, which abrogate the proliferative capacity of the tumor cells while maintaining the ability of the tumor cells to trigger antigen-specific and costimulatory signals in T cells and thus to stimulate an immune response.

The tumor-based immunotherapeutic cells can be administered to the subject by injection of the tumor cells into the subject. The route of injection can be, for example, intravenous, intramuscular, intraperitoneal or subcutaneous. Administration of the tumor-based immunotherapeutic cells at the site of the original tumor may be beneficial for inducing T cell-mediated immune responses against the original tumor. Administration of the tumor-based immunotherapeutic cells in a disseminated manner, e.g. by intravenous injection, may provide systemic anti-tumor immunity and, furthermore, may protect against metastatic spread of tumor cells from the original site. The modified'primary immune-privilege tumor cells can be administered to a subject prior to or in conjunction with other forms of therapy or can be administered after other treatments such as chemotherapy or surgical intervention.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., immunovaccines of the present invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by, and directly dependent on, the unique characteristics of the active compound, the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention further contemplates compositions containing immunovaccines in combination with a small molecule(s). For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the scope of knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such polypeptides may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and Thorpe et al. “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The above described modulating agents may be administered it the form of expressible nucleic acids which encode said agents. Such nucleic acids and compositions in which they are contained, are also encompassed by the present invention. For instance, the nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### V. Methods of Prevention, Prognosis and Treatment

The present invention provides for both prophylactic and therapeutic methods of treating a subject, e.g., a human, who has or is at risk of (or susceptible to) cancer, e.g., ovarian cancer. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, inhibiting, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” or “compound” includes, but is not limited to, cells, small molecules, peptides, peptidomimetics, polypeptides, miRNA, RNA interfering agents, e.g., siRNA molecules, antibodies, ribozymes, and antisense oligonucleotides.

One aspect of the invention pertains to methods for treating a subject suffering from cancer (e.g., ovarian cancer). These methods involve administering to a subject a composition of the present invention (e.g., immunovaccine) which upregulates the subject's immune response to the cancer cells. The immunovaccines of the present invention can be used alone or in appropriate association, as well as in combination, with other pharmaceutically active compounds as are known in the art.

The subject to which the compositions of the present invention (e.g., immunovaccine) are administered is preferably a mammal such as a non-primate (e.g., cows, pigs, horses, cats, dogs, rats etc.) and a primate (e.g., monkey such as a cynomolgous monkey and a human). In some embodiments, the subject is a human.

In other various embodiments, the methods and compositions of the present invention are used to prevent, prognose, or treat cancer (e.g., ovarian cancer) in which a therapeutic or prophylactic immunoreactive reagent is useful for treatment or prophylaxis. In one embodiment, the cancer (e.g., ovarian cancer) is amenable to prevention, prognosis, or treatment by an enhanced immune response, more preferably an infectious disease, cancer or a neurodegenerative or amyloid disorder.

The compositions can be utilized for the prevention of a variety of cancers, e.g., in individuals who are predisposed as a result of familial history or in individuals with an enhanced risk to cancer due to environmental factors, for the prevention of infectious diseases, e.g., in individuals with enhanced risks of exposure to agents of infectious disease, and for the prevention of neurodegenerative or amyloid diseases, for example in individuals with genetic predispositions to neurodegenerative or amyloid diseases.

The methods and compositions of the invention may be used in patients who are treatment naive, in patients who have previously received or are currently receiving treatment with a composition of the present invention, in patients who have previously received or are currently receiving treatment with an immunoreactive reagent, or in patients who have previously received or are currently receiving treatment with other pharmaceutical agents or combinations, including but not limited to anti-cancer agents, antibiotics, anti-bacterial agents, anti-fungal agents and anti-viral agents. In one embodiment, a composition of the invention (e.g., immunovaccine) is administered to a patient that has previously received or is currently receiving treatment with immunotherapeutic reagents. In another embodiment, a composition of the invention (e.g., immunovaccine) is administered to a patient that has previously received or is currently receiving treatment with such a composition of the invention. In yet another embodiment of the invention, a composition of the invention (e.g., immunovaccine) is administered to a patient that has previously received or is currently receiving treatment that includes, but is not limited to, anti-cancer agents, antibiotics, anti-bacterial agents, anti-fungal agents or anti-viral agents, optionally with an immunoreactive reagent. In still another embodiment, a composition of the invention (e.g., immunovaccine) is administered to a patient that has previously received or is currently receiving treatment that includes, but is not limited to, anti-cancer agents, antibiotics, anti-bacterial agents, anti-fungal agents or anti-viral agents, optionally with a composition of the invention (e.g., immunovaccine).

The methods and compositions of the invention may also be used to treat patients that have previously received treatment with a composition of the invention (e.g., immunovaccine) or immunoreactive reagents and are currently not efficiently treated with respect to each treatment administered alone.

In some embodiments, the methods and compositions of the invention can be used to diagnose or prognose a subject as having or at risk of developing a cancer (e.g., ovarian cancer). Thus, the present invention provides a method for identifying such cancer with increased or reduced numbers of a composition of the invention (e.g., immunovaccines comprising engineered tumor cells) in which the composition of the invention is administered to the subject at a first timepoint, a test sample is obtained from the subject at a later time point, and the amount of engineered tumor cells administered at the first time point is detected, wherein an increase in the amount of, engineered tumor cells indicates a negative or poor prognosis and a decrease or stable amount of engineered tumor cells indicates a positive or good prognosis. As used herein, a “test sample” refers to a biological sample obtained from a subject of interest. For example, a test sample can be a biological fluid as defined above.

A skilled artisan will understand that the amount of engineered tumor cells can be detected and determined in a number of ways. In one embodiment, the representative amount of engineered tumor cells in a biological sample (e.g., biopsy or biological fluid sample) can be determined. In other embodiments, detection of a detectable label (e.g., luciferase) that is proportional to the amount of engineered tumor cells can be determined. For example, subjects can be administered a diagnostically-effective amount of engineered tumor cells comprising a detectable label at the onset of treatment, and this value can be compared to that of a sample obtained at a later time point.

Furthermore, the prognostic assays described herein can be used to monitor the influence of compositions of the invention (e.g., immunovaccines) during clinical trials. For example, the effectiveness of compositions of the invention (e.g., immunovaccines) as described herein to treat cancer (e.g., ovarian cancer) can be monitored in clinical trials of subjects exhibiting indications of treated cancer as described herein (e.g., by determining the amount of engineered tumor cells remaining in a subject at a time point after the initial time point of administration. In this way, the composition of the invention can serve as a marker, indicative of the physiological response of the subject to the composition of the invention. Accordingly, this response state may be determined before, and at various points during treatment of the individual with the composition of the invention.

The present invention further encompasses methods for preventing, prognosing, or treating a cancer or metastasis in a subject comprising in any order the steps of administering to the subject a composition of the invention (e.g., immunovaccine) in a therapeutically effective dose and manner.

In certain embodiments, the compositions and methods of the invention can be used to prevent, inhibit or reduce the growth or metastasis of cancerous cells. In a specific embodiment, the administration of a composition of the invention (e.g., immunovaccine) inhibits or reduces the growth or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth or metastasis in absence of the administration of composition.

Cancers that can be treated according to the methods of the invention include, but are not limited to, leukemia (e.g., acute leukemia such as acute lymphocytic leukemia and acute myelocytic leukemia), neoplasms, tumors (e.g., non-Hodgkin's lymphoma, fibrosarcoma, myxosarcoma, liposarcoma, chondrosarcoma, osteogenic sarcoma, chordoma, angiosarcoma, endothehosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease (B-cell lymphoma), metastases, or any disease or disorder characterized by uncontrolled cell growth. In certain embodiments, the cancer is ovarian cancer.

Tumor antigens or tumor associated antigens include mesothelin, MUC-1, CA-125, GM-CSF, HER-2/neu, folate binding protein, cancer-germ cell (CG) antigens (MAGE, NY-ESO-1), mutational antigens (MUM-1, p53, CDK-4), over-expressed self-antigens (p53, HER2/NEU), viral antigens (from Papilloma Virus, Epstein-Barr Virus), tumor proteins derived from non-primary open reading frame mRNA sequences (Y-ES01, LAGE1), Melan A, MART-1, MAGE-1, MAGE-3, BAGE, GAGE-1, GAGE-2, tyrosinase, gp100, gp75, c-erb-B2, CEA, PSA, Stn, TAG-72, KSA (17-1A), PSMA, p53 (point mutated and/or overexpressed), RAS (point mutated), EGF-R, VEGF, GD2, GM2, GD3, Anti-Id, CD20, CD19, CD22, CD36, Aberrant class II, B1, CD25 (IL-2R) (anti-TAC), or HPV.

In one embodiment, a method or composition of the invention is used for treating or preventing a cancer or metastasis in a subject comprising the administration of a composition of the invention (e.g., immunovaccine) in combination with a standard therapeutic known in the art for the treatment of cancer (e.g., ovarian cancer). For example, a composition of the invention (e.g., immunovaccine) can be administered in combination with, but not limited to, paclitaxel, cisplatin, carboplatin, cytokines (e.g., intereferon gamma and interleukin-2), chemotherapy, and radiation treatment. Such conjunctive therapies also comprise immunostimulants. An immunostimulant refers to essentially any substance that enhances or potentiates an immune response (antibody and/or cell-mediated) to an exogenous antigen. One type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

This invention is further illustrated by the following examples which should not be construed as limiting. The contents of all references, patents and published patent applications cited throughout this application, as well as the Figures, are incorporated herein by reference.

### EXAMPLES

### Example 1

### Materials and Methods Used in Examples 2-9

A. Mice

Female C57BL/6 mice were acquired from the National Cancer Institute. Female CD40^{+/+} (B6.129P2-CD40^{tmlKir}/J) mice, TLR4^{lps-del} (C57BL/10ScNJ) mice, and TLR2^{-/-} (TLR^{tmlKir}/TLR2^{tmlKir}, B6.129-TLR2^{tmlKir}/J) mice were purchased from The Jackson Laboratory. All animals were maintained under specific pathogen-free conditions, and all procedures were done according to approved protocols and in accordance with recommendations for the proper use and care of laboratory animals.

B. Plasmid DNA Constructs and DNA Preparation

For generation of retroviral plasmids encoding murine secretory Hsp70-T2A peptide (sHsp70)-green fluorescent protein (GFP) and the control T2A peptide-GFP, murine Hsp70 was first cloned into pSecTag2 B® (Invitrogen) by PCR cloning using the forward primer 5′-CCCAAGCTTATGGCCAAGAACACGGCGAT-3′ containing a HindIII enzyme site and the backward primer 5′-CGGGATCCATCCACCTCCTCGATGGTGG-3′ containing a BamHI site. The sequences between NheI and BamHI, which contains one murine immunoglobulin κ-chain signal peptide fused with Hsp70, were subcloned into the Nod (blunted) and BamHI sites of a retroviral vector pMSCV-FLAG. Two complementary oligonucleotides encoding Thosea asigna virus 2A peptide EGRGSLLTCGDVEENPGP (Szymczak et al. (2004) Nat. Biotech. 22, 589-594) containing BamHI site on one and EcoRI site on the other were synthesized, annealed, and cloned into the corresponding sites of pMSCV-FLAG. Enhanced GFP (EGFP) gene was inserted between EcoRI and XhoI. The control plasmid pMSCV-T2A-GFP consists of the same arrangements of genes but devoid of sHsp70. A retroviral construct pLuci-thy1.1 expressing both luciferase and thy1.1 was reported in Hung et al. (2007) Gene Ther. 14, 20-29. The amplified luciferase cDNA was inserted into the BglII and HpaI sites of the bicistronic vector pMIG-thy1.1. Both luciferase and thy1.1 cDNA are under the control of a single promoter element and separated by internal ribosomal entry site. All of the constructs were verified by restriction analysis and DNA sequencing using ABI 3730 DNA Analyzer®.

C. Cell Lines

The MOSEC and TC-1 cell lines were generated as in Roby et al. (2000) Carcinogenesis 21, 585-591 and Lin et al. (1996) Cancer Res. 56, 21-26. The MOSEC cell line was originally derived from mouse ovarian surface epithelial cells as described in Roby et al. (2000) Carcinogenesis 21, 585-591. The TC-1 cell line was generated by in vitro culture of primary lung epithelial cells and transduction with HPV-16 E6 and E7 transformative genes, which immortalized the cells, as well as the c-Ha-ras oncogene (Lin et al. (1996) Cancer Res. 56, 21-26). MOSEC/luc or TC-1-luciferase (TC-1/luc) cells were generated as described in Hung et al. (2007) Vaccine 25, 127-135. For stable expression of sHsp70-GFP and GFP on these two cell lines, pMSCV-FLAG/sHsp70-T2A-GFP or GFP was transfected into Phoenix® packaging cell line using LipofectAMINE® (Invitrogen) and the virion-containing supernatant was collected 48 hours after transfection. The supernatant was then filtered through a 0.45-mm cellulose acetate syringe filter (Nalgene) and used to infect MOSEC/luc cells in the presence of 8 mg/mL polybrene (Sigma). Transduced cells were isolated using preparative flow cytometry of stained cells with GFP signal sorting. The growth rate of MOSEC/luc (or TC-1/luc)/Hsp70-GFP cells was comparable with those of MOSEC/luc (or TC-1/luc)/GFP cells.

D. Western Blot

To detect Hsp70 protein expression in the culture medium and cells, 1×10⁵ MOSEC/luc/sHsp70-T2A-GFP and MOSEC/luc/GFP cells were seeded in six-well plate. Forty-eight hours after seeding the cells, medium from culture was collected and cells were lysed with protein extraction reagent (Pierce). Equal amounts of proteins (10 μg) or medium (30 μL) were loaded and separated by SDS-PAGE using a 10% polyacrylamide gel. The gels were electroblotted to a polyvinylidene difluoride membrane (Bio-Rad Laboratories). Blots were blocked with PBS/0.05%, Tween 20 (TTBS) containing 5% nonfat milk for 2 hours at room temperature. Membranes were probed with rabbit anti-Hsp70 antibody (StressGen) for 1 h, washed four times with TTBS, and then incubated with sheep anti-mouse IgG conjugated to horseradish peroxidase (Amersham) at 1:1,000 dilution in TTBS containing 5% nonfat milk. Membranes were washed four times with TTBS and visualized under ChemiDoc XRS chemiluminescent detection system (Bio-Rad Laboratories).

E. Tumorigenesis Assay

Naive C57BL/6 mice were challenged i.p. with 1×10⁶ live TC-1/luc/sHsp70-GFP and TC-1/luc/GFP or 1×10⁶ MOSEC/luc/sHsp70-GFP and MOSEC/luc/GFP. CD40^{-/-}, TLR4^{lps-del}, and TLR2^{-/-} mice were challenged with 1×10⁶ live MOSEC/luc/sHsp-GFP and MOSEC/luc/GFP cells. Detection of luminescence activity indicating relative tumor loading was done by Xenogeny IVIS200® Imaging System on a weekly basis.

F. Tumor Protection Assay

Naive C57BL/6 mice were i.p. injected with 1×10⁶ live or irradiated MOSEC/luc/GFP cells and MOSEC/luc/sHsp70-GFP cells. The irradiated MOSEC/luc/GFP or MOSEC/luc/sHsp70-GFP tumor cells were prepared using an irradiation dosage of 90,000 cGy/10 min. Luciferase activity was checked 2 weeks later. For those mice in which tumor luminescent activities have declined by 2 weeks (except live MOSEC/luc/GFP group), 1×10⁶ MOSEC/luc cells were used to i.p. challenge again 2 weeks after vaccination. Differences in the luminescence activity of tumor growth were monitored once weekly.

G. Tumor Treatment

C57BL/6 mice were i.p. injected with 1×10⁶ MOSEC/luc cells. After 5 days, mice were treated with irradiated 1×10⁶ MOSEC/luc/GFP or MOSEC/luc/sHsp70-GFP cells. Differences in the luminescence activity of tumor growth were monitored once weekly.

H. Depletion of Lymphocyte Subsets In Vivo

Those mice vaccinated with irradiated 1×10⁶ MOSEC/luc/sHsp-GFP or MOSEC/luc/GFP cells were injected i.p. with blocking antibody using a protocol similar to one as described in Chen et al. (2000) Cancer Res. 60, 1035-1042. Mice were injected with 100 μg of purified rat monoclonal antibody (mAb) GK1.5 (anti-CD4), 2.43 (anti-CD8), and PK136 (anti-NK1.1). Depletion was started 1 week after cell-based vaccination and continued every other day for the first week and then once every week. Depletion was assessed 1 day after the third administration of antibodies and 1 day after the fourth administration of antibodies by flow cytometry analysis of spleen cells stained with 2.43, GK1.5, or PK136. It was found that >90% depletion of CD8, CD4, or NK cells was achieved. These mice were challenged with MOSEC/luc tumor cells 2 weeks after vaccination. Depletion was maintained by continuing the antibody injections weekly for the duration of the tumor imaging follow-up. Differences in the luminescence activity of tumor growth were monitored once weekly.

I. Intracellular Cytokine Staining and Flow Cytometry Analysis

Mice were vaccinated with 1×10⁶ irradiated MOSEC/luc/Hsp70-GFP or MOSEC/luc/GFP or 1×10⁶ irradiated TC-1/luc/Hsp70-GFP or TC-1/luc/GFP cells twice at 1-week interval. Splenocytes were harvested from mice 1 week after the last vaccination. Pooled splenocytes (5×10⁶) from each vaccination group were incubated for 7 days with 1 μg/mL murine mesothelin peptide (for MOSEC cell lines, amino acids 406-414; Hung et al. (2007) Gene Ther. 14, 921-929) or with no peptide as control. For TC-1 cell lines, pooled splenocytes were stimulated with 1 murine E7 peptide (amino acids 49-57; Feltkamp et al. (1993) Eur. J. Immunol. 23, 2242-2249) or no peptide overnight directly. Cell surface marker staining for CD8 and intracellular cytokine staining for IFN-γ as well as flow cytometry analysis were done under conditions as described in Chen et al. (2000) Cancer Res. 60, 1035-1042. Analysis was done on a Becton Dickinson FACScan® with CellQuest® software (Becton Dickinson Immunocytometry System). Each group was measured in triplicate and data were shown as mean±SD in numerical bar.

J. Characterization of In Vitro and Serum Concentrations of Hsp70

Mouse Hsp 70 cDNA was amplified using forward primer 5′-gggatccATGGCCAAGAACACGGCGAT-3′ and backward primer 5′-CCGCTCGAGctaatccacctcctcgatggt-3′ in a polymerase chain reaction (PCR) and the amplified product was then cut with BamHI and XhoI enzymes. The amplified product was then cloned into pGEX-6p-1 vector which contained GST protein (GE Healthcare, Pittsburgh, Pa.). pGEX-Hsp70 vector was then introduced into E. coli BL21 (DE3). The transfected bacterial cells were grown at 37° C. in Magic Media® (Invitrogen, Carlsbad, Calif.) overnight. GST-tagged Hsp70 protein was extracted by standard lysis protocol, and purified using GSTrap HP® column.

For serum Hsp70 protein detection, groups of mice (3 per group) were injected with 1×10⁶ or 2×10⁷ Mosex-luc/sHsp70-GFP or Mosec-luc/GFP cells intraperitoneally. Serum samples were taken on D0, D3, and D7. Pertoneal washing samples were collected on D3 and D7 by performing peritoneal lavage with 1 ml of PBS, washing and recovering the peritoneal fluid. Culture supernatants were collected after 48-hour culture of 80% confluence of above cells. Hsp70 protein concentrations in culture medium, sera and peritoneal lavage were determined by enzyme-linked immunosorbent assay (ELISA) using mouse monoclonal Hsp70 antibody (Stressgen, BC, Canada, SPA-810) as capture antibody and rabbit polyclonal Hsp70 antibody (SPA-812) as the detection antibody. In brief, capture antibody (2 μg/ml) was coated on a 96-well microtiter styrene plate and incubated at 4° C. overnight. The next day, wells were blocked with blocking buffer (0.1 M NaHCO₃, [pH8.6], 5 mg/ml BSA, 0.02% NaN₃) for 2 hours. Hsp70 purified protein from bacteria and serum samples were diluted 4-fold in serum diluent (Immunochemistry Technologies, Bloomington, Minn.). Those serially diluted purified Hsp70 protein and sera containing Hsp70 from each injection were added into the well and incubated for 2 hours. After washing with PBS 0.5% Tween, wells were added with detection antibody (1 μg/ml) and incubated for one hour, followed by HRP-conjugated anti-rabbit secondary antibody (Amersham Bioscience, Little Chalfont, UK) and 1-Step Turbo TMB® substrate (Pierce, Rockford, Ill.) as the standard protocol.

K. Statistical Analysis

All data expressed as the mean±standard deviation (SD) are representative of at least two different experiments. Comparisons between individual data points were made using a Student's t test. Differences in survival between experimental groups were analyzed using the Kaplan-Meier approach. The statistical significance of group differences will be assessed using the log-rank test.

### Example 2

### Cells Transduced with Retrovirus Encoding Hsp70-GFP Express the Secreted Form of the Mouse Hsp70 Protein

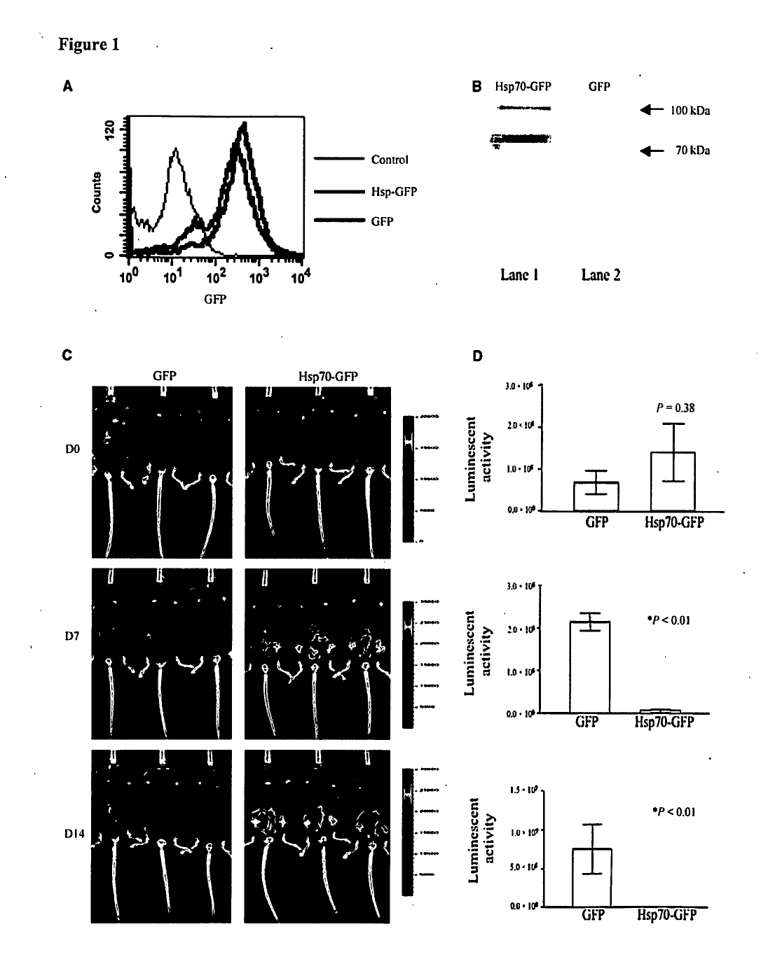

Retrovirus encoding sHsp70-T2A-GFP (referred to as Hsp70-GFP) or T2A-GFP (referred to as GFP) were generated. The GFP expression in cells allowed the distinguishing of transfected cells from untransfected cells. Furthermore, T2A is a self-cleavage peptide from T asigna virus that cleaves cotranslationally and allowed determination of the effect of secreted Hsp70 (Szymczak et al. (2004) Nat. Biotech. 22, 589-594). To characterize whether MOSEC/luc cells transduced with retrovirus encoding Hsp70-GFP or GFP express comparable levels of the gene encoded by the retrovirus, flow cytometry analysis was performed for GFP expression. As shown in FIG. 1A, comparable levels of GFP expression were observed in both the MOSEC/luc cells transduced with Hsp70-GFP and MOSEC/luc cells transduced with GFP. To further determine if MOSEC/luc cells transduced with retrovirus encoding Hsp70-GFP led to secretion of the mouse Hsp70 protein in the culture medium, Western blot analysis was performed using the supernatant from cultured MOSEC/luc cells transduced with Hsp70-GFP or GFP. As shown in FIG. 1B, the supernatant of MOSEC/luc cells transduced with Hsp70-GFP contained a 70-kDa protein, consistent with the secreted form of mouse Hsp70 protein, as well as an ˜100-kDa protein, which represents the uncleaved fusion protein of sHsp70 and EGFP. The total amount of secreted Hsp70 from irradiated MOSEC/luc/sHsp70-GFP cells in culture using the ELISA was also determined. Purified recombinant Hsp70 protein from bacteria was used to generate a standard curve. The concentration of Hsp70 from the supernatant of 1×10⁶ of irradiated MOSEC/luc/sHsp70-GFP cells seeded on the culture dish for 24 hours was found to be 74.36±2.87 ng/mL. Because the whole amount of the supernatant was 2 mL, the total amount of Hsp70 protein secreted from 1×10⁶ of irradiated MOSEC/luc/sHSp70-GFP cells in 24 hours was 148.72 ng. Thus, the data indicate that MOSEC/luc cells transduced with Hsp70-GFP express the secreted form of Hsp70 protein.

### Example 3

### Mice Challenged with MOSEC/Luc Cells Expressing Hsp70-GFP Fail to Develop Tumor Growth

The in vivo tumor growth in mice challenged with MOSEC/luc cells expressing Hsp70-GFP or GFP was subsequently tested. The tumor growth of the challenged mice was characterized using bioluminescent imaging systems. As shown in FIG. 1C, the mice challenged with MOSEC/luc cells expressing Hsp70-GFP showed a significant reduction in luciferase activity over time. In contrast, the mice challenged with MOSEC/luc cells expressing GFP showed increased luciferase activity over time. The luciferase activity of the tumor-challenged mice was quantified in the form of bar graphs (FIG. 1D). The data indicate that viable MOSEC/luc cells expressing Hsp70-GFP failed to grow in tumor-challenged mice. The in vitro proliferation rate and in vivo growth rate in nude mice of MOSEC/luc cells expressing Hsp70-GFP and MOSEC/luc cells expressing GFP was also characterized and no significant difference in proliferation was found. Thus, the fact that MOSEC/luc cells expressing Hsp70-GFP failed to grow in tumor-challenged mice was not due to differences in proliferation of MOSEC/luc cells expressing Hsp70-GFP and MOSEC/luc cells expressing GFP or to the toxicity of transfection of cells with GFP.

Furthermore, an ELISA was performed to determine the serum levels of secreted Hsp70 in vaccinated mice. Purified recombinant Hsp70 protein from bacteria was used to generate a standard curve. Mice were vaccinated with MOSEC/luc/sHsp70-GFP or MOSEC/luc/GFP (control) cells at doses of 1×10⁶ or 2×10⁷ cells per mouse. Sera from vaccinated mice were taken on days 0, 3, and 7. It was found that Hsp70 was only detectable after injection of MOSEC/luc/sHsp70-GFP cells at a dose of 2×10⁷ cells per mouse on day 3 (see FIG. 7A-7C). The concentration of the serum Hsp70 was determined to be 18.17±4.3 ng/mL. Because Hsp70 is bound to scavenger receptors such as CD91 (Srivastava (2002) Annu. Rev. Immunol. 20, 395-425), which are commonly expressed in macrophages and other types of cells in vivo, the secreted Hsp70 may be easily absorbed from the serum, resulting in low serum levels. Thus, it is difficult to detect serum Hsp70 unless large amounts of MOSEC/luc/sHsp70-GFP cells were injected.

### Example 4

### Mice Previously Challenged with MOSEC/Luc Cells Expressing Hsp70-GFP Generate Long-Term Protective Antitumor Effects Against MOSEC/Luc and Prolonged Survival

To determine if the mice previously challenged with MOSEC/luc cells expressing Hsp70-GFP generate long-term antitumor effects against MOSEC/luc, in vivo tumor protection experiments were performed. The previously challenged mice were rechallenged i.p. with MOSEC/luc cells. Naive mice were also challenged with MOSEC/luc as a control. The tumor growth of the MOSEC/luc cells in challenged mice was monitored using bioluminescent imaging systems. As shown in FIG. 2A, the mice previously challenged with MOSEC/luc cells expressing Hsp70-GFP showed a significant reduction in luciferase activity over time. In contrast, the naive mice challenged with MOSEC/luc cells showed increased luciferase activity over time. The luciferase activity of the tumor-challenged mice was quantified in the form of bar graphs, as shown in FIG. 2A. These data indicate that the mice previously challenged with MOSEC/luc cells expressing Hsp70-GFP generated long-term protective antitumor effects against MOSEC/luc cells. The survival of tumor-challenged mice was further characterized using the Kaplan-Meier survival analysis. As shown in FIG. 2B, prolonged survival was observed in mice previously challenged with MOSEC/luc cells expressing Hsp70-GFP compared with naive mice control. Taken together, the data indicate that mice previously challenged with MOSEC/luc cells expressing Hsp70-GFP generate a long-term protective antitumor effect and prolonged survival.

### Example 5

### Mice Immunized with Irradiated MOSEC/Luc Cells Expressing Hsp70-GFP Show Significant Decrease in Tumor Load and Prolonged Survival

For clinical translation, it is important to use irradiated tumor cell-based vaccines instead of live tumor cell-based vaccines. Thus, in vivo tumor protection experiments were performed using irradiated MOSEC/luc cells expressing Hsp70-GFP. C57BL/6 mice were immunized i.p. with 1×10⁶ per mouse of irradiated MOSEC/luc cells expressing either Hsp70-GFP or GFP. The irradiated MOSEC/luc/GFP or MOSEC/luc/sHsp70-GFP tumor cells were prepared by using an irradiation dosage of 90,000 cGy/10 min. Two weeks later, the mice were challenged with MOSEC/luc cells. The tumor growth of the challenged mice was characterized using bioluminescent imaging systems. As shown in FIG. 2C, the mice immunized with irradiated MOSEC/luc expressing Hsp70-GFP showed a significant reduction in luciferase activity over time. In contrast, the mice immunized with irradiated MOSEC/luc cells expressing GFP showed increased luciferase activity over time. The luciferase activity was quantified in the form of bar graphs, as shown in FIG. 2C. These data indicate that immunization with irradiated MOSEC/luc cells expressing Hsp70-GFP generates a protective antitumor effect. The survival of the tumor-challenged mice were further analyzed using Kaplan-Meier survival analysis. As shown in FIG. 2D, prolonged survival in mice immunized with irradiated MOSEC/luc cells expressing Hsp70-GFP was observed compared with mice immunized with irradiated MOSEC/luc cells expressing GFP. Taken together, the data indicate that immunization with irradiated MOSEC/luc cells expressing Hsp70-GFP generates a protective antitumor effect and prolongs survival.

### Example 6

### Mice Challenged with TC-1/Luc Cells Expressing Mouse Hsp70-GFP Show Slow Development of Tumor Growth and Longer Survival

To determine whether the effect observed in mice immunized with MOSEC/luc tumor cells expressing Hsp70-GFP is applicable to other tumor models, the tumor growth in the TC-1 tumor cell line was observed. TC-1 is a highly potent tumor cell line and expresses highly specialized tumor antigens. The C57BL/6 mice were challenged with viable TC-1/luc cells expressing either Hsp70-GFP or GFP and were characterized using bioluminescent imaging systems. A significant reduction in luciferase activity was observed over time in the mice challenged with TC-1/luc cells expressing Hsp70-GFP. The luciferase activity was quantified in the form of bar graphs (FIG. 8A). Thus, the data indicate that viable TC-1/luc cells expressing Hsp70-GFP showed slow tumor growth in challenged mice similar to what is observed in the case of MOSEC/luc cells expressing Hsp70-GFP. Furthermore, when mice were immunized with irradiated TC-1/luc cells expressing Hsp70-GFP, the vaccinated mice also generated potent protective antitumor effects.

### Example 7

### Mice Vaccinated with Irradiated Tumor Cells Expressing Hsp70-GFP Generate Significantly Higher Frequency of Activated Antigen-Specific CD8⁺ T Cells

To determine the antigen-specific CD8⁺ T-cell immune responses in mice vaccinated with irradiated tumor cells expressing Hsp70-GFP, flow cytometry analyses were performed to determine the number of antigen-specific IFN-γ-secreting CD8⁺ T cells using splenocytes from vaccinated mice. C57BL/6 mice were vaccinated i.p. with either TC-1/luc cells expressing Hsp70-GFP or GFP (FIGS. 3A-3B) or MOSEC/luc cells expressing Hsp70-GFP or GFP (FIGS. 3C-3D). Because the TC-1 has been shown to express HPV-16 E7 and MOSEC cells have been shown to express mesothelin, E7 or mesothelin-specific CD8⁺ T-cell immune response in mice vaccinated with irradiated TC-1 cells expressing Hsp70-GFP or irradiated MOSEC/luc cells expressing Hsp70-GFP, respectively, were analyzed. Splenocytes from vaccinated mice were stimulated with either E7- or mesothelin-specific peptides. The E7-specific antigenic peptide (amino acids 49-57; Feltkamp et al. (1993) Eur. J. Immunol. 23, 2242-2249) and the mesothelin-specific peptide (amino acids 406-414; Hung et al. (2007) Gene Ther. 14, 921-929) have been characterized as a MHC class I-restricted CD8⁺ T-cell epitope in C57BL/6 mice. Thus, these peptides allow for the characterization of the E7- or mesothelin-specific immune response in vaccinated mice. As shown in FIGS. 3A and 3C, significantly higher number of antigen-specific IFN-γ-secreting CD8⁺ T cells was observed in mice vaccinated with irradiated tumor cells expressing Hsp70-GFP compared with mice vaccinated with irradiated tumor cells expressing GFP. A graphical representation of the number of IFN-γ⁺ CD8⁺ T cells is depicted in FIGS. 3B and 3D. The data indicate that mice vaccinated with irradiated tumor cells expressing Hsp70-GFP are capable of generating a potent antigen-specific CD8⁺ T-cell immune response.

### Example 8

### CD8⁺, NK, and CD4⁺ Cells are Important for Protective Antitumor Effect Generated by Irradiated Tumor Cell-Based Vaccines Expressing Hsp70-GFP

To determine the major subset of lymphocytes important for the protective antitumor effect observed in mice vaccinated with irradiated MOSEC/luc cells expressing Hsp70-GFP, in vivo antibody depletion experiments were performed using monoclonal antibodies (mAbs) specific for CD4⁺ T cells, CD8⁺ T cells, or NK cells. C57BL/6 mice were vaccinated with irradiated MOSEC/luc expressing Hsp70-GFP. Mice vaccinated with irradiated MOSEC/luc cells expressing GFP without lymphocyte depletion were used as a control. Two weeks after vaccination, the mice were challenged with MOSEC/luc cells. Depletion was initiated 1 week before tumor challenge. Tumor growth was monitored using bioluminescent imaging systems. As shown in FIG. 4A, high luciferase activity in Hsp70-GFP-vaccinated mice depleted of CD8⁺, NK, or CD4⁺ cells was observed compared with the vaccinated mice without depletion. A graphical representation of the luminescent activity data is depicted in FIG. 4B. Thus, the data indicate that the CD8⁺, NK, and CD4⁺ cells are important for protective antitumor immunity observed in mice vaccinated with irradiated MOSEC/luc cells expressing Hsp70-GFP.

### Example 8

### Vaccination with Irradiated MOSEC/Luc Cells Expressing Hsp70-GFP Generates a Significant Therapeutic Antitumor Effect and Promotes Long-Term Survival

To test the therapeutic effects of treatment with irradiated MOSEC/luc cells expressing Hsp70-GFP, C57BL/6 mice were challenged i.p. first with MOSEC/luc cells and then treated them 5 days later with irradiated MOSEC/luc cells expressing either Hsp70-GFP or GFP. Tumor growth in tumor-challenged mice was then monitored using bioluminescent imaging systems. As shown in FIG. 5A, significant reduction in luciferase activity in mice treated with irradiated MOSEC/luc cells expressing Hsp70-GFP was observed over time. In comparison, the tumor-challenged mice treated with irradiated MOSEC/luc cells expressing GFP showed an increase in luciferase activity over time. A graphical representation of the luciferase activity data is depicted in FIG. 5B. These data indicate that treatment with irradiated MOSEC/luc cells expressing Hsp70-GFP leads to significant therapeutic antitumor effect. The survival of the treated mice was also analyzed using the Kaplan-Meier survival analysis. As shown in FIG. 5C, prolonged survival in mice treated with irradiated MOSEC/luc cells expressing Hsp70-GFP were observed compared with mice treated with irradiated MOSEC/luc cells expressing GFP. Thus, the data indicate that treatment with irradiated MOSEC/luc cells expressing Hsp70-GFP leads to significant therapeutic antitumor effect and prolonged survival.

### Example 9

### CD40 and TLR4 Receptors are Important for Inhibiting In Vivo Tumor Growth of the Viable MOSEC/Luc Expressing Hsp70-GFP

It has also been implicated that CD40, TLR2, and TLR4 (MaSsa et al. (2004) Cancer Res. 64, 1502-1508; Sanchez-Perez et al. (2006) J. Immunol. 177, 4168-4177; Theriault et al. (2005) FEBS Lett. 579, 1951-1960; Whittall et al. (2006) Eur. J. Immunol. 36, 2304-2314; Wang et al. (2001) Immunity 15, 971-983; Asea et al. (2002) J. Biol. Chem. 277, 15028-15034; Becker et al. (2002) J. Cell Biol. 158, 1277-1285) can bind with Hsp70 and are important for Hsp70-mediated immune adjuvant effects. To determine if these molecules are important for inhibiting in vivo tumor growth of the viable MOSEC/luc expressing Hsp70-GFP, in vivo tumor growth was analyzed in CD40, TLR2, or TLR4 knockout C57BL/6 mice. The mice were challenged with 1×10⁶ per mouse of viable MOSEC/luc cells expressing Hsp70-GFP. Naive mice were included as a control. The tumor growth of the challenged mice was characterized using bioluminescent imaging systems and luciferase activity was quantified in the form of bar graphs (FIG. 6). As shown in FIG. 6, the naive mice and TLR2 knockout mice challenged with MOSEC/luc cells expressing Hsp70-GFP showed a significant reduction in tumor growth (luciferase activity) over time. In contrast, the CD40 knockout mice challenged with MOSEC/luc cells expressing GFP showed the most significant increase in tumor growth (luciferase activity) over time. The TLR4 knockout mice challenged with MOSEC/luc cells expressing GFP showed moderate increase in tumor growth (luminescent activity). The data indicate that viable MOSEC/luc cells expressing Hsp70-GFP failed to grow in tumor-challenged naive and TLR2 knockout mice but did grow largely in CD40 and minimally in TLR4 knockout mice. Thus, CD40 is the most important protein, followed by TLR4, in the mechanism of the inhibiting tumors expressing Hsp70-GFP.

Thus, Hsp70-secreting murine ovarian cancer cells (MOSEC) have been created that express luciferase. It was found that mice challenged with MOSEC/luc cells expressing Hsp70-GFP generate significant mesothelin-specific CD8⁺ T-cell immune responses and significant therapeutic effect against MOSEC/luc cells. Furthermore, the same approach is applicable to other tumor models, such as E7-expressing TC-1 tumor cell models. In addition, it has been shown that the protective antitumor effect is mainly contributed to by CD8⁺, NK, and CD4⁺ cells. It was also found that CD40 and TLR4 receptors are important for inhibiting in vivo tumor growth of the viable MOSEC/luc expressing Hsp70. It has been shown herein that the use of the noninvasive bioluminescence imaging systems serves as great tool for characterizing the tumor load over time.

In addition, significant enhancement of antigen-specific immune response in mice vaccinated with irradiated tumor cells secreting Hsp70 was observed. There are several properties of Hsp70 that may contribute to the generation of antigen-specific CD8⁺ T-cell immune responses. For example, Hsp70 has been shown to bind with antigenic peptides and is capable of binding with CD91 receptor on antigen-presenting cells (Srivastava (2002) Annu. Rev. Immunol. 20, 395-425). Furthermore, Hsp has been shown to facilitate cross-presentation of bound antigenic peptide (Noessner et al. (2002) J. Immunol. 169, 5424-5432; Li et al. (2002) Curr. Opin. Immunol. 14, 45-5; Chen et al. (2004) J. Leukoc. Biol. 75, 260-266). Moreover, Hsp is capable of activating dendritic cells (Flohe et al. (2003) J. Immunol. 170, 2340-2348). Thus, a combination of these factors significantly contributes to the generation of antigen-specific immune responses generated by tumor cells secreting Hsp70.

Furthermore, CD40 was observed to be the most important for inhibiting tumor growth of MOSEC/luc cells expressing Hsp70-GFP. CD40 is an extracellular receptor for binding and uptake of Hsp70-peptide complexes (Becker et al. (2002) J. Cell Biol. 158, 1277-1285). The binding of Hsp70-peptide complexes from tumor cells with CD40 may facilitate the cross-presentation of tumor-antigenic peptides by antigen-presenting cells. Furthermore, binding of Hsp70-peptide complexes to antigen-presenting cells that express CD40 may also lead to activation of dendritic cells, resulting in secretion of proinflammatory cytokines via p38/nuclear factor-κB signaling pathway (Becker et al. (2002) J. Cell Biol. 158, 1277-1285). Thus, the CD40 molecule is crucial for the inhibition of the growth of tumor cells expressing Hsp70-GFP.

As a result, the newly created MOSEC/luc tumor model will serve as an important model for the characterization of tumor load and distribution in tumor-challenged mice using noninvasive bioluminescence imaging. Previous studies also validate the use of bioluminescence imaging system for quantitatively measuring tumor load in vivo (Tseng et al. (2004) Nat. Biotech. 22, 70-77; Tseng et al. (2004) Cancer Res. 64, 6684-6692; Jenkins et al. (2003) Clin Exp. Metastasis 2003, 20:733-44; Drake et al. (2005) Clin. Exp. Metastasis 22, 674-84). Tumor load was also analyzed by gross examination of the peritoneal cavity and it was found that the tumor volume correlates with the intensity of the luminescence imaging. Furthermore, the luminescence activity correlated well with mouse survival rate. Thus, the bioluminescence imaging used in the present Examples represents a plausible noninvasive approach to measure tumor load and distribution in mice.

### Example 10

### Vaccination of Human Subjects with Engineered Tumor Cells

The compositions and methods described herein can be applied to human tumor cells (e.g., human ovarian cancer cell line, OVCAR3 available from the ATCC) for vaccine development applicable to human subjects. For example, OVCAR3 expresses several known ovarian tumor antigens, such as CA125, folate receptor, MUC1, and mesothelin. It also expresses NYESO-1, one of the most immunogenic known antigens. Thus, tumor cell-based vaccines using OVCAR3 cell line may be used to generate common ovarian tumor antigen-specific immune responses resulting in significant antitumor effects against a majority of ovarian carcinomas.

A. Generation and Characterization of High-Grade Serous Carcinoma Cell Line that Stably Secretes High Levels of Hsp70

To generate OVCAR3 expressing secreted human heat shock protein 70 (NCBI accession NM_{—}005345.4), the pNGVL4a-sHsp70 construct can be used to stably transfect OVCAR3 cells using lipofectamine 2000 (Invitrogen). The gene vector pNGVL4a has been in several human clinical trials. In order to construct secreted heat shock protein, human Hsp70 is first cloned into a pSecTag2 B vector (Invitrogen) by standard PCR cloning procedures using the forward primer 5-CCCAAGCTTATGGCCAAAGCCGCGGCGAT-3 containing a HindIII enzyme site and the reverse primer 5-CCCGGATCCCTAATCTACCTCCTCAATGG-3 containing a BamHI site. The sequences between NheI and BamHI, containing one Ig k-chain signal peptide fused with Hsp70, is subcloned into the EcoRV (blunted) and BamHI sites of pNGVL4a to generate pNGVL4a-sHsp70 according to the following, wherein the red color sequences are Ig k-chain leader sequences for the secreted signal and the black color sequences are human hsp70 sequences.

| 10 | 20 | 30 | 40 | 50 | 60 | 70 | 80

| | | |
|---|---|---|
| 1 | ATGGAGACAG ACACACTCCT GCTATGGGTA CTGCTGCTCT GGGTTCCAGG TTCCACTGGT GACGCGGCCC AGCCGGCCAG | 80 |
| 81 | GCGCGCGCGC CGTACGAAGC TTatggccaa agccgcggcg atcggcatcg acctgggcac cacctactcc tgcgtggggg | 160 |
| 161 | tgttccaaca cggcaaggtg gagatcatcg ccaacgacca gggcaaccgc accaccccca gctacgtggc cttcacggac | 240 |
| 241 | accgagcggc tcatcgggga tgcggccaag aaccaggtgg cgctgaaccc gcagaacacc gtgtttgacg cgaagcggct | 320 |
| 321 | gatcggccgc aagttcggcg acccggtggt gcagtcggac atgaagcact ggcctttcca ggtgatcaac gacggagaca | 400 |
| 401 | agcccaaggt gcaggtgagc tacaaggggg acaccaaggc attctacccc gaggagatct cgtccatggt gctgaccaag | 480 |
| 481 | atgaaggaga tcgccgaggc gtacctgggc tacccggtga ccaacgcggt gatcaccgtg ccggcctact tcaacgactc | 560 |
| 561 | gcagcgccag gccaccaagg atgcgggtgt gatcgcgggg ctcaacgtgc tgcggatcat caacgagccc acggccgccg | 640 |
| 641 | ccatcgccta cggcctggac agaacgggca agggggagcg caacgtgctc atctttgacc tgggcggggg caccttcgac | 720 |
| 721 | gtgtccatcc tgacgatcga cgacggcatc ttcgaggtga aggccacggc cggggacacc cacctgggtg gggaggactt | 800 |
| 801 | tgacaacagg ctggtgaacc acttcgtgga ggagttcaag agaaaacaca agaaggacat cagccagaac aagcgagccg | 880 |
| 881 | tgaggcggct gcgcaccgcc tgcgagaggg ccaagaggac cctgtcgtcc agcacccagg ccagcctgga gatcgactcc | 960 |
| 961 | ctgtttgagg gcatcgactt ctacacgtcc atcaccaggg cgaggttcga ggagctgtgc tccgacctgt tccgaagcac | 1040 |
| 1041 | cctggagccc gtggagaagg ctctgcgcga cgccaagctg gacaaggccc agattcacga cctggtcctg gtcgggggct | 1120 |
| 1121 | ccacccgcat ccccaaggtg cagaagctgc tgcaggactt cttcaacggg cgcgacctga acaagagcat caaccccgac | 1200 |
| 1201 | gaggctgtgg cctacggggc ggcggtgcag gcggccatcc tgatggggga caagtccgag aacgtgcagg acctgctgct | 1280 |
| 1281 | gctggacgtg gctcccctgt cgctggggct ggagacggcc ggaggcgtga tgactgccct gatcaagcgc aactccacca | 1360 |
| 1361 | tccccaccaa gcagacgcag atcttcacca cctactccga caaccaaccc ggggtgctga tccaggtgta cgagggcgag | 1440 |
| 1441 | agggccatga cgaaagacaa caatctgttg gggcgcttcg agctgagcgg catccctccg gcccccaggg gcgtgcccca | 1520 |
| 1521 | gatcgaggtg accttcgaca tcgatgccaa cggcatcctg aacgtcacgg ccacggacaa gagcaccggc aaggccaaca | 1600 |
| 1601 | agatcaccat caccaacgac aagggccgcc tgagcaagga ggagatcgag cgcatggtgc aggaggcgga gaagtacaaa | 1680 |
| 1681 | gcggaggacg aggtgcagcg cgagagggtg tcagccaaga acgccctgga gtcctacgcc ttcaacatga agagcgccgt | 1760 |
| 1761 | ggaggatgag gggctcaagg gcaagatcag cgaggccgac aagaagaagg tgctggacaa gtgtcaagag gtcatctcgt | 1840 |
| 1841 | ggctggacgc caacaccttg gccgagaagg acgagtttga gcacaagagg aaggagctgg agcaggtgtg taaccccatc | 1920 |
| 1921 | atcagcggac tgtaccaggg tgccggtggt cccgggcctg ggggcttcgg ggctcagggt cccaagggag ggtctgggtc | 2000 |
| 2001 | aggccccacc attgaggagg tagattag | 2028 |
| | \| 10 \| 20 \| 30 \| 40 \| 50 \| 60 \| 70 \| 80 | |

All of the constructs are then verified by restriction analysis and DNA sequencing using standard techniques.

B. Characterization of sHsp70 by ELISA and Western Blot

For the detection of sHsp70 protein concentration in OVCAR3 cells, an indirect ELISA is performed according to Chang et al. (2007) Cancer Res. 67, 10047-10057. sHsp70 transfected OVCAR3 cells are then seeded in 96-well plate. Medium from culture is subsequently collected according to various times after seeding. Medium is then serially diluted in PBS, coated in a 96-microwell plate, and incubated at 4° C. overnight. The wells are then blocked with PBS containing 20% fetal bovine serum. After washing with PBS containing 0.05% Tween-20, the plate is incubated with a 1/1000 dilution of rabbit anti-Hsp70 antibody (StressGen, Victoria, British Columbia) for 2 hours at 37° C. The plate is then further incubated with 1/1000 dilution of a peroxidase-conjugated donkey anti-rabbit IgG antibody (Amersham Pharmacia, Piscataway, N.J.) at room temperature for one hour. The plate is then washed, developed with 1-STEP™ Turbo TMB-ELISA (Pierce, Rockford, Ill.) and stopped with 1M H₂SO₄. The concentration of Hsp70 protein is determined by comparison to a standard curve of purified Hsp70 protein. ELISA measurements of intensity are made on three replicate samples from each pool and the mean and standard error are reported. The intensity of Hsp70 proteins is plotted over time and the results are compared with a standard curve of purified Hsp70 protein. The OVCAR3 clones expressing high levels of sHsp70 are isolated and cloned twice by limiting dilutions. The stability of the clone and its expression of sHsp70 are monitored. The expression of sHsp are also validated by Western blot according to Chang et al. (2007) Cancer Res. 67, 10047-10057.

C. cGMP Manufacture and Release of Master Cell Banks and a Clinical Lot, per FDA CBER Guidelines, of Hsp70-Secreting Ovarian Cancer Cell-Based Vaccines

Cloned, mycoplasma-free OVCAR3 cell lines that stably-secrete human hsp70 (OVCAR3-sHSP70) are used to optimize growth media, cell seeding/harvesting, cell scale-up from, for example, T-150 and T-225 flasks, to Corning ten-stack cell trays, rapid cell concentration, cryopreservation, HLA typing, cell potency, post-thaw viability, Hsp-70 secretion, ELISA, and assessment of surface mesothelin levels by flow cytometry (Thomas et al. (2004) J. Exp. Med. 200, 297-306) per FDA guidelines for good laboratory practice (GLP) assays (i.e., Sterility and Bacteriostasis & Fungistasis testing, Mycoplasma Test, Bacterial EndoToxin/Limulus Ameobocyte Lysate: Kinetic/chromogenic Assay Cell Line Species Identity by Isoenzymes, Thin Section Electron Microscopy—Cell Morphology and Virus Detection/Tabulation, PCR-based Product-Enhanced Reverse Transcriptase Assay, In Vitro Assay for Detection of Adventitious Viruses, In Vivo Assay for Viral Contaminants, Detection of Adventitious Bovine Viruses, Detection of HIV-1 DNA, Detection of EBV DNA, Detection of HTLV I and II DNA, Detection of Parvovirus B19 DNA, Detection of HIV-II DNA, Detection of AAV-2 DNA, Detection of HHV-7 DNA, Detection of HHV-8 DNA, Detection of CMV DNA, Detection of HCV DNA, and Detection of HHV-6 DNA). In addition, the most cost-effective manner for optimal production of safe and potent Master Cell Bank of 1-3×10⁹ cells is performed.

An irradiation study is also performed with OVCAR3-sHSP70 cells irradiated 10,000, 15,000 and 20,000 rads and subsequently cultured over a minimal thirty-five day period, to ensure that the appropriate irradiation dose results in 100% cell growth arrest. In addition, a pilot run(s) at ¼ scale of the anticipated clinical production run, while the actual OVCAR3-sHSP70 Master Cell Bank is performed to ensure a robust, reproducible cost-effective methodology that is optimized for scale up to a 1-2×10¹⁰ cell clinical lot. This pilot process development study includes “hold” steps and lot release safety testing in order to mimic the actual process and timing required for the final harvest, irradiation, and vial fill finish of 1-2×10¹⁰ bulk cells.

Quality assurance review of all documentation including batch production records, environmental monitoring records and all internal quality control and GLP release tests result in the issuance of certificates of analysis and certificates of cGMP conformance for the clinical lot.

D. Phase I Clinical Studies Using Clinical Grade Hsp70-Secreting Ovarian Cancer Cell-Based Vaccine in Patients with High-Grade Serous Carcinoma

With the cGMP-grade reagent available, a phase I clinical trial is performed in patients with high-grade ovarian serous carcinoma to evaluate the safety, feasibility, and immunogenicity of a clinical grade OVCAR3-sHSP70 vaccine. The study population for this trial comprises patients with advanced high-grade serous carcinoma (stage III/IV) who have completed standard chemotherapy with minimal residual disease, but have high risk for recurrence. After obtaining informed consent, candidate subjects are screened for eligibility. Patients identified as potential candidates for treatment are screened for HIV and Hepatitis testing and consent forms are acquired before screening. The following eligibility criteria are also satisfied:
1) Patients are 18 or older and have a histopathologically confirmed diagnosis of stage III/IV ovarian serous carcinoma. Patients with high grade serous carcinoma of the peritoneum (primary peritoneal carcinoma) of fallopian tube are also eligible.
2) Patients are HIV negative.
3) Patients are not pregnant. All patients with the potential for pregnancy and/or fertility are to use acceptable birth control methods.
4) Patients are to have a GOG performance grade of <1.
5) Patients are to have recovered from the effects of recent surgery, radiotherapy or chemotherapy. At least four weeks are to have elapsed between study entry and the completion of prior chemotherapy or radiotherapy. Two weeks are to have elapsed since any surgery.
6) Patients are to have adequate bone marrow, hepatic and renal function:
   ANC≧1,500/μL,
   platelets ≧100,000/μL;
   total bilirubin ≦1.5 mg/dL
   SGOT, SGPT, and alkaline phosphatase ≦2.5× institutional normal creatinine ≦2 mg/dL.
7) Patients are to have no active infections and have a life expectancy of at least 12 weeks.
8) Patients are to have no medical problems unrelated to the malignancy of sufficient severity to limit full compliance with the study or expose them to undue risk. Patients who have an active autoimmune disease or who are receiving immunosuppressive medications that result in significant systemic levels of suppression, including corticosteroids, are not eligible. However, nasal steroids steroid suppositories are allowed.
9) Patients are not to have other active malignancy.
10) No other experimental therapies are intended to treat the patient's malignancy.
11) Patients are to give informed consent according to federal, state, and institutional guidelines indicating that they are aware of the investigational nature of the study.
12) All patients eligible for this study are to be presented at a gynecologic oncology tumor board and must be discussed with the principal investigator and be approved by the principal investigator before study entry.

The following exclusion criteria are to be used to invalidate prospective patients:
1) Diagnosis of immunosuppressive disease or use of immunosuppressive medication.
2) Infection by HIV, Hepatitis B or C.
3) Presence of uncontrolled intercurrent illness including, but not limited to, ongoing or active infection, symptomatic congestive heart failure, unstable angina pectoris, untreated or new cardiac arrhythmia, or psychiatric illness/social situations that would limit compliance with study requirements.
4) Presence or history of autoimmune disease that has required treatment in the past or for which the subject is currently receiving treatment.
5) Pregnancy or breast feeding.
6) History of prior malignancy is allowed if patient has been disease free for >5 years.
7) Patients with known CNS metastases are excluded.
8) Inability to understand or unwillingness to sign an informed consent document.

A higher number of patients are screened than the number of patients than will be enrolled in the trial, since some of the patients may not meet the requirements for the trial. Patients have blood samples drawn before treatment to determine whether ovarian tumor antigen-specific CD8⁺ T cells can be detected in peripheral blood at baseline. Blood is collected every 2 weeks after initial immunization in order to determine if vaccination can generate ovarian tumor antigen-specific CD8⁺ T cell responses. Antigen-specific T cells are analyzed pre- and post-immunization for mesothelin, folate receptor, MUC-1 and NYESO-1-specific immune responses. These proteins are commonly expressed by the majority of ovarian serous carcinoma.

The following also described additional evaluations and measurements:
Tumor staging and imaging: Clinical staging are based on physical exam by an experienced gynecologic oncologist and imaging studies (computed tomography or magnetic resonance imaging studies, as clinically indicated). Staging is performed by use of criteria established by the American Joint Committee on Cancer, Cancer Staging Manual, Fifth Edition, 1997. Extent of measurable or evaluable disease is documented before therapy, at the completion of therapy and at defined intervals after therapy is completed.
Performance status: Performance status is based on the Gynecologic Oncology Group performance status scale ranging from 0 to 4.
Eligibility testing: The testing described above is provided to screen patients for pre-existing conditions that may place them at increased risk for toxicity from vaccination. These tests include HIV serology, hepatitis B virus surface antigen, surface antibody and core antibody and hepatitis C serology, and 13HCG for women of childbearing age.
Serum chemistry panel: The comprehensive chemistry panel includes total bilirubin, alkaline phosphatase, alanine aminotransferase, aspartate aminotransferase, albumin, and total protein. In addition, phosphate, direct bilirubin, magnesium and uric acid are measured.
Electrolytes/BUN/CR: Sodium, potassium, bicarbonate, chloride, blood urea nitrogen, serum creatinine, glucose, magnesium and calcium.
CBC with differential: Hematocrit, hemoglobin, platelet count, white blood cell count with neutrophil, basophil, mononuclear, lymphocyte and eosinophil counts are analyzed.
Urinalysis: Includes evaluation of pH, protein, heme, glucose and microscopic analysis for WBC and RBC.
ECG: Electocardiogram is performed during pretreatment evaluation to establish a baseline and is to be repeated, if clinically indicated.
Toxicity testing: These tests screen vaccine recipients for muscle inflammation and generation of anti-DNA antibodies in response to vaccination. Tests include ESR, CPK, LDH.
DTH: Delayed-type hypersensitivity testing is to be performed with subcutaneous administration of candida albicans, tetanus toxoid, mumps and trichophyton antigens. The site of administration is carefully marked and recorded. The site is examined 48 hours after placement and the reaction to the type of each antigen is recorded as maximal diameter of induration in millimeters.
Immunology samples: During the weeks of vaccine administration, samples are drawn prior to receipt of vaccination. Five, 10 cm³ green top (sodium heparin) tubes of peripheral blood are delivered immediately after phlebotomy for peripheral blood mononuclear cell preparations via Ficoll-Hypaque density gradient centrifugation according to standard protocol and stored at −70° C. until further analysis. Cells are characterized for in vitro ovarian tumor antigen-specific CD8⁺ T cell responses and their cytokine profiles following stimulation with the various overlapping ovarian tumor antigens. PBMCs from patients with HLA-A2.1 are characterized for HLA-A2.1 restricted ovarian tumor antigen-specific CD8⁺ T cell precursor activity with peptide-loaded HLA-A.2 tetramers. One, 7 cm³ lavender top (EDTA) tube is to be sent to a flow cytometry lab for CD4/CD8 count during the pretreatment evaluation, week 16 and week 20.
HLA typing: One 10 cm³ green top (sodium heparin) tube of peripheral blood is submitted for HLA typing. Determination of the patient's HLA type is important for immunologic monitoring of response, to vaccination.
Vaccination: Irradiated OVCAR3-sHSP70 vaccine as described herein.
Disease status assessment: Clinical disease status is assessed by physical examination (which may include pelvic examination), CA-125 serology and imaging studies, if indicated. Response is classified by the new international criteria proposed by the Response Evaluation Criteria in Solid Tumors (RECIST), as further described herein.
Vaccination site assessment: Patients have the site of vaccination examined for pain, erythema, warmth, tenderness, induration, hematoma and purpura. Patients are evaluated 24, 48 and 72 hours after the first vaccination and weekly after subsequent vaccinations or as otherwise clinically indicated.
Adverse event evaluation: Patients are evaluated for side effects of therapy by the principal investigator, research nurse or other designated personnel. Toxicity is graded per the Cancer Therapy Evaluation Program Common Toxicity Criteria (CTC) version 2.0.
Concomitant medication review: All medications taken by the patient, including over the counter preparations, is noted at each clinical visit.
Follow-up: Patients return to the clinic two weeks after their most recent vaccination. They are then seen every two weeks through week 26 unless there is documentation of disease recurrence. Patients not receiving other therapy and/or have no evidence of recurrence are followed after week 26 and are seen every 12 weeks up to a maximum of 4 years after treatment. Patients who recur or progress and require alternate therapy before week 26 are not required to return for follow-up after progression, although clinical follow-up is encouraged.

Vaccine is administered 4 times by intradermal injections of irradiated OVCAR3-sHSP70 vaccine with 4-week intervals after patients have completed the standard chemotherapy with minimal residual disease. The intradermal administration approach is chosen as the method for delivering the vaccine because the skin has significant numbers of Langerhans cells, which are immature dendritic cells that function to pick up antigen for effective antigen processing and presentation to T cells. Extensive preclinical data demonstrates that the intradermal approach provides a superior route. Furthermore, it has been shown that intradermal administration of the Hsp70-secreting irradiated ovarian cancer cells generated compatible levels of ovarian tumor antigen-specific CD8⁺ T cell immune responses compared to intraperitoneal administration of the tumor cell-based vaccine. Finally, extensive clinical experience exists with intradermal administration of other cell-based vaccines including irradiated tumor cell-based vaccines that secrete GM-CSF. An accelerated titration design is used for dose escalation (since minimal toxicity is anticipated) from a starting dose of 1×10⁷ irradiated OVCAR3-sHSP70 vaccine up to 3×10⁸ irradiated OVCAR3-sHSP70 cells based on extensive pre-clinical and clinical data showing that doses between 1×10⁸ and 3×10⁸ irradiated vaccine cells are feasible to administer and showed evidence of bioactivity and the induction of antigen-specific T cell responses (Jaffee et al. (2001) J. Clin. Oncol 0.19, 145-156). A standard dose escalation approach is used according to Table 1.

TABLE 1

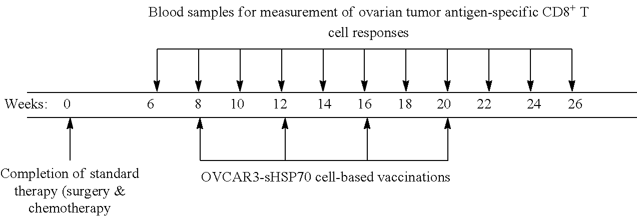

| Vaccine | Number of | Route of | | Number of |
|---|---|---|---|---|
| Dose | vaccinations | administration | Regimen | patients |
| 1 x 10⁷ | 4 | intradermal | Wks 8, 12, 16, 20 | 3 |
| 3 x 10⁷ | 4 | intradermal | Wks 8, 12, 16, 20 | 3 |
| 1 X 10⁸ | 4 | intradermal | Wks 8, 12, 16, 20 | 3 |
| 3 X 10⁸ | 4 | intradermal | Wks 8, 12, 16, 20 | 9* |

*The maximum tolerated or feasible dose will be expanded to 9 patients if no major toxicity is observed in the initial 3 patients.

The 4 dose cohorts studied include: 1×10⁷, 3×10⁷, 1×10⁸, and 3×10⁸ OVCAR3-sHSP70 vaccine cells administered intradermally. Three patients each receive a total dose of either 1×10⁷, 3×10⁷ 1×10⁸ or 3×10⁸ cells per vaccination. Assuming no significant toxicity is observed in the maximum dose, 6 additional patients are added to the maximum dose cohort (3×10⁸). Thus, the maximum number of patients in the trial is 18. For each patient, the total number of immunizing cells injected during each vaccination period remains the same throughout the series of vaccinations. Each dose is divided into three (1×10⁷, 3×10⁷ or 1×10⁸ vaccine cells) or six (3×10⁸ cells) 0.6 ml aliquots, and each aliquot is delivered intradermally into the right and left thighs and the non-dominant arm. Patients receiving 3×10⁸ cells receive two aliquots of 0.6 ml per limb.

The dose and regimen is based on previous clinical trial using allogeneic GM-CSF cell-based vaccines (Jaffee et al. (2001) J. Clin. Oncol 0.19, 145-156). OVCAR3-sHSP70 is administered with 4-week intervals between doses, with up to four doses per subject. For determination of dose limiting toxicity (DLT), at least 3 subjects and up to 9 subjects are enrolled in each dose group. Related (possibly or probably related) DLTs is defined on the basis of adverse events observed between the time of the first dose administration through 21 days after the first dose as defined by the Cancer Therapy Evaluation Program Common Toxicity criteria (CTC) version 3.0. Subjects are replaced if they do not receive a second dose of vaccine due to disease progression or circumstances unrelated to the study. Subjects are enrolled in the next higher dose cohort after at least two subjects at a given dose level are followed for at least 7 days after administration of their first dose, with no more than 0/3 or 1/6 subjects experiencing a DLT that is possibly or probably related to the OVCAR3-sHSP70 vaccine. Dose-limiting toxicity is defined as any grade 3 or 4 toxicity occurring on or after the first day of vaccine administration based on the CTC for toxicity and event reporting. The maximally tolerated dose refers to the dose of irradiated OVCAR3-sHSP70 ovarian cancer cell-based vaccine that results in ≦1 of 9 patients to experience a dose limiting toxicity (DLT). This is the highest dose level below the maximally administered dose. The maximally administered dose refers to the dose which produces ≧1 of 3 patients to experience a DLT or the dose which produces ≧1 of 9 patients at a given dose level to experience a DLT. The maximally feasible dose is determined by both the maximal volume that can be administered intradermally without discomfort (two cubic centimeters) and the maximal concentration of irradiated OVCAR3-sHSP70 ovarian cancer cell-based vaccine in a directly injectable starch-based cryopreservant, saline (e.g., 3×10⁸ cells per patient). Table 2 summarizes the schedule of evaluations and measurements for the clinical trial.

TABLE 2

| | Completion of | | | | | | | | | | | | |
|---|---|---|---|---|---|---|---|---|---|---|---|---|---|
| | standard therapy | | | | | | | | | | | | |
| | & Pre-study | Wk | Wk | Wk | Wk | Wk | Wk | Wk | Wk | Wk | Wk | Month | Off |
| | Wk 0-6 | 8 | 10 | 12 | 14 | 16 | 18 | 20 | 22 | 24 | 26 | 9-48 | study |
| OVCAR3- | | X | | X | | X | | X | | | | | |
| sHSP70 | | | | | | | | | | | | | |
| Vaccination^{A} | | | | | | | | | | | | | |
| Informed | X | | | | | | | | | | | | |
| consent | | | | | | | | | | | | | |
| Demographics | X | | | | | | | | | | | | |
| Medical history | X | | | | | | | | | | | | |
| Class I and II | X | | | | | | | | | | | | |
| HLA typing | | | | | | | | | | | | | |
| Physical exam | X | X | X | X | X | X | X | X | X | X | X | X | X |
| Vital signs | X | X | X | X | X | X | X | X | X | X | X | X | X |
| Height | X | | | | | | | | | | | | |
| Weight | X | X | X | X | X | X | X | X | X | X | X | X | X |
| Performance | X | X | X | X | X | X | X | X | X | X | X | X | X |
| status | | | | | | | | | | | | | |
| CBC w/diff, plts | X | X | X | X | X | X | X | X | X | X | X | X | X |
| Serum | X | X | X | X | X | X | X | X | X | X | X | X | X |
| chemistry^{B} | | | | | | | | | | | | | |
| Adverse event | X | X-------------------------------------------------------------------------------------------------------------------------------X | | | | | | | | | | | |
| evaluation | | | | | | | | | | | | | |
| Characterization | X | Patients undergo phlebotomy for ovarian tumor antigen-specific T cells testing as baseline and weekly after | | | | | | | | | | | |
| of ovarian tumor | | initial vaccination. Further characterization of antigen-specific CD8+ T cells includes enzyme-linked | | | | | | | | | | | |
| antigen-specific | | immunospot (ELISPOT) assay, intracellular cytokine staining with flow cytometry analysis, and peptide-loaded | | | | | | | | | | | |
| CD8+ T cells | | HLA-A.2 tetramer staining for cases with known CTL epitope. Maintenance of CTL responses is evaluated by | | | | | | | | | | | |
| | | repeat testing each month for a maximum of 6 months. | | | | | | | | | | | |
| Imaging studies | X | Radiologic measurements are performed every 12 weeks after completion of vaccination for documentation of | | | | | | | | | | | |
| (CT, chest | | time to disease progression for a maximum of 4 years. | | | | | | | | | | | |
| imaging) | | | | | | | | | | | | | |
| B-HCG^{C} | X | | | | | | | | | | | | |
| HIV | X | | | | | | | | | | | | |
| Tumor antigen- | X | | | | | | X | | | | | | |
| specific ELISA | | | | | | | | | | | | | |
| CPK, ESR | X | | X | X | X | X | X | | | | | | |
| CD4 and CD8 | X | | | | | | X | | | | | | |
| count | | | | | | | | | | | | | |
| CA125 levels | X | X | X | X | X | X | X | X | X | X | X | X | X |

^{A}Dose as assigned
^{B}Albumin, alkaline phosphatase, total bilirubin, bicarbonate, BUN, calcium, chloride, creatinine, glucose, LDH, phosphorus, potassium, total protein, SGOT [AST], SGPT [ALT], sodium.
^{C}Serum pregnancy test (women of childbearing potential).

The immunological data is used to correlate with the clinical outcomes. Based on the trial design, valuable clinical specimens are generated, such as PBMCs and serum, which allow assessment of the ovarian tumor antigen-specific immune responses before and after vaccination.

E. Characterization of Tumor Antigen-Specific CD8+ T Cell Immune Responses in Vaccinated Individuals

Patients are monitored with physical examination, disease imaging, and molecular analyses. For disease imaging, conventional CT techniques are used with cuts of 10 mm or less in slice thickness contiguously. Spiral CT should be performed using a 5 mm contiguous reconstruction algorithm. This applies to the chest, abdomen, and pelvis. In some instances, disease may be best imaged using PET/CT. Standard FDG-glucose administration and imaging will be used.

The various ovarian antigen-specific CD8⁺ T cell immune responses are analyzed using quantitative ovarian antigen specific CD8⁺ T cell immunological assays, including ELISPOT, intracellular cytokine staining followed by flow cytometry analysis, and peptide-loaded MHC class I tetramer staining. PBMCs are collected before vaccination, during vaccination, and after vaccination. For mesothelin, folate receptor, and NY-ESO-1, 10 15-mers overlapped by 10 amino acids or HLA-A2 specific epitopes are used to stimulate PBMC and assay various ovarian tumor antigen specific CD8+ T cell immune responses (listed in Table 3). For MUC1, a variable number of tandem-repeated 20-amino acid segments VNTR motif (PDTRPAPGSTAPPAHGVTSA) of MUC1 or HLA-A2 specific epitopes for CD8 specific T cell assays are used. For CA125 specific CD8⁺ T cell immune responses, dendritic cells pulsed with 500 U CA125 protein (Sigma Aldrich, St. Louis, Mo.) or 500 U CA125 protein and 5 ug/ml CA125 antibody (Invitrogen) immuno-complex are used to stimulate PBMC and assay CA125 CD8 specific T cell responses as described in Schultes and Whiteside (2003) J. Immunol. Methods 279, 1-15.

An IFN-g ELISPOT assay is performed using an IFN-g ELISPOT assay kit from Mabtech Inc (Cincinnati, Ohio) using the methods similar to those described in Peng et al., (2007) Clin. Cancer Res. 13, 2479-2487. A positive control is included using PBMCs stimulated with PHA (Cat. No. 30852801, Remel Inc., Lenexa, Kans.) and/or CEF peptides. Wells without any peptides and without PBMC and peptides are used as negative controls. The captured IFN-g are detected with biotin-conjugated anti-human IFN-g monoclonal antibody (Clone 7-B6-1) and followed by incubation with horseradish peroxidase (HRP)-conjugated streptavidin (Cat. No. 3310-9). The forming spots are developed by adding Avidin-Enzyme-Complex (Cat. No. SK-4200, Vector Laboratories, Burlingame, Calif.) and stopped by washing with tap water. The number of spots are analyzed on an ELISPOT Analyzer 3B (Cellular Technology Ltd., Cleveland, Ohio).

Intracellular cytokine staining assay are performed using the methods similar to those described in Peng et al. (2007) Clin. Cancer Res. 13, 2479-2487 and Hung et al. (2007) Vaccine 25, 127-135. As positive controls, HiCK-1 Cytokine Positive Control cells (for IL-2) and HiCK-2 Cytokine Positive Control cells (for IL-4 and IL-10) are used. Cells are surface stained with PE-conjugated anti-CD8. The cells are then permeabilized and fixed with Cytofic/Cytoperm (BD Pharmingen, San Diego, Calif.) and stained for intracellular cytokines with FITC-conjugated anti-IFN-g, anti-IL-2, anti-IL-4, APC-conjugated anti-IL-10 and anti-TNF-a. Flow cytometry analysis is performed using FACSCalibur with CELLQuest software (BD Biosciences, Mountain View, Calif.).

Peptide-loaded MHC class I tetramer assays are performed using methods similar to those described in Hung et al. (2007) Gene Therapy 14, 921-929. PBMCs (1×10⁶) from HLA-A2 patients are incubated with various HLA-A2 peptides (listed in Table 3) loaded tetramers (Beckman Coulter, San Diego, Calif., USA) on ice followed by PE-conjugated goat anti-mouse IgG1 and murine monoclonal anti-human CD8-FITC (BD) applied to identify CD8⁺ lymphocytes. Fluorometric analysis are performed on a FACScan (Becton Dickinson) and lymphocytes are analyzed using CELLQuest software (Becton Dickinson).

TABLE 3

| | Protein or overlapping | HLA-A2 | |
|---|---|---|---|
| Antigen | peptides | epitopes | Reference: |
| Mesothelin | Overlapping peptide | 20-28 | Hung et al. (2007) |
| | | SLLFLLFSL | Vaccine 25, 127-135; |
| | | 530-538 | Thomas et al. (2004) |
| | | VLPLTVAEV | J. Exp. Med. 200, |
| | | 540-549 | 297-306; Yokokawa |
| | | KLLGPHVEGL | et al. (2005) Clin. |
| | | | Cancer Res. 11, 6342-6351 |
| Folate receptor | Overlapping peptide | 191-199 | Peoples et al. (1999) |
| | | EIWTHSTKV | Clin. Cancer Res. 5, |
| | | 24-253 | 4214-4223 |
| | | LLSLALMLL | |
| NY-ESO-1 | Overlapping peptide | 157-165 | Jager et al. (1998) J. |
| | | SLLMWITQC | Exp. Med. 187, 265-270 |
| MUC1 | VNTR motif: | 950-958 | Brossart et al. (1999) |
| | PDTRPAPGSTAPPAHGVTSA | STAPPVHNV | Blood 93, 4309-4317 |
| | | 12-20 | |
| | | LLLLTVLTV | |

The data generated from characterizing the ovarian tumor antigen specific immune responses is used to correlate with the clinical outcomes of the patients receiving vaccination with the Hsp70-secreting tumor cell-based vaccine. A critical component of testing these cancer vaccines is the characterization and monitoring of cellular immunological parameters serving as direct indicators of effective vaccination. These cellular immunological parameters are then correlated with antitumor effects in a quantitative manner. The immunological parameters are compared with quantitative antitumor effect data using regression analysis to determine which parameters are the most critical indicators of a potent vaccine effect. For example, the immunological parameter(s) that display the best correlation with the number of peritoneal tumor nodules represent a desirable indicator for predicting antitumor effects. This information allows for a determination of which parameters are the most critical indicators of a potent vaccine effect.

The difference between baseline ovarian tumor antigen-specific CTL levels and CTL levels at the different time points following vaccination are analyzed by use of the paired t-test or the Wilcoxon signed rank sum test, whichever is appropriate given observed data. Data is also analyzed for the presence of overall trends using a random effects linear longitudinal data model which accounts for correlated observations from the same individual over time. T cells are modeled as a function of time. The within patient variability is of certain interest in planning for additional studies. As such, after fitting a linear model which sufficiently describes the average effect of standard therapy on T cell response over the course of standard therapy, residuals (i.e. the difference between the observed data and the fitted model) between within and across patients is calculated. From these residuals, the variance is estimated in measures from an individual over time and also the variance in T cells between two individuals at a given time.

Assuming the tumor cell-based vaccines have minimal toxicity and favorable early phase clinical trials, the tumor cell-based vaccines are combined with other cancer vaccines to generate innovative combination immunotherapeutic strategies against high-grade ovarian serous carcinoma. Any adverse events defined as any untoward medical occurrence in a patient or clinical investigation subject administered an investigational product regardless of causality assessment are reported according to the CTC for toxicity and event reporting.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A tumor cell-based vaccine comprising tumor cells that are genetically modified to constitutively express at least one heat shock protein.

2-33. (canceled)


SEQUENCE LISTING (18 sequences)

SEQ ID NO: 1 - protein, 4 aa, Artificial Sequence
  Description of Artificial Sequence Synthetic peptide
KDEL

SEQ ID NO: 2 - DNA, 29 bp, Artificial Sequence
  Description of Artificial Sequence Synthetic primer
cccaagcttatggccaagaacacggcgat

SEQ ID NO: 3 - DNA, 28 bp, Artificial Sequence
  Description of Artificial Sequence Synthetic primer
cgggatccatccacctcctcgatggtgg

SEQ ID NO: 4 - protein, 18 aa, Thosea asigna virus
EGRGSLLTCGDVEENPGP

SEQ ID NO: 5 - DNA, 27 bp, Artificial Sequence
  Description of Artificial Sequence Synthetic primer
gggatccatggccaagaacacggcgat

SEQ ID NO: 6 - DNA, 30 bp, Artificial Sequence
  Description of Artificial Sequence Synthetic primer
ccgctcgagctaatccacctcctcgatggt

SEQ ID NO: 7 - DNA, 29 bp, Artificial Sequence
  Description of Artificial Sequence Synthetic primer
cccaagcttatggccaaagccgcggcgat

SEQ ID NO: 8 - DNA, 29 bp, Artificial Sequence
  Description of Artificial Sequence Synthetic primer
cccggatccctaatctacctcctcaatgg

SEQ ID NO: 9 - DNA, 2028 bp, Artificial Sequence
  Description of Artificial Sequence Synthetic polynucleotide
atggagacagacacactcctgctatgggtactgctgctctgggttccaggttccactggtgacgcggcccagccggccaggcgcgcgcgccgtacgaagcttatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggacaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattag

SEQ ID NO: 10 - protein, 20 aa, Artificial Sequence
  Description of Artificial Sequence Synthetic peptide
PDTRPAPGSTAPPAHGVTSA

SEQ ID NO: 11 - protein, 9 aa, Artificial Sequence
  Description of Artificial Sequence Synthetic peptide
SLLFLLFSL

SEQ ID NO: 12 - protein, 9 aa, Artificial Sequence
  Description of Artificial Sequence Synthetic peptide
VLPLTVAEV

SEQ ID NO: 13 - protein, 10 aa, Artificial Sequence
  Description of Artificial Sequence Synthetic peptide
KLLGPHVEGL

SEQ ID NO: 14 - protein, 9 aa, Artificial Sequence
  Description of Artificial Sequence Synthetic peptide
EIWTHSTKV

SEQ ID NO: 15 - protein, 9 aa, Artificial Sequence
  Description of Artificial Sequence Synthetic peptide
LLSLALMLL

SEQ ID NO: 16 - protein, 9 aa, Artificial Sequence
  Description of Artificial Sequence Synthetic peptide
SLLMWITQC

SEQ ID NO: 17 - protein, 9 aa, Artificial Sequence
  Description of Artificial Sequence Synthetic peptide
STAPPVHNV

SEQ ID NO: 18 - protein, 9 aa, Artificial Sequence
  Description of Artificial Sequence Synthetic peptide
LLLLTVLTV